(19) United States
(12) Patent Application Publication
(10) Pub. No.: US 2021/0076962 A1
(43) Pub. Date: Mar. 18, 2021

(54) APPARATUS, SYSTEMS AND METHODS FOR DIAGNOSING PARKINSONS DISEASE FROM ELECTROENCEPHALOGRAPHY DATA

(71) Applicant: University of Iowa Research Foundation, Iowa City, IA (US)

(72) Inventors: Soura Dasgupta, Iowa City, IA (US); Kumar Narayanan, Iowa City, IA (US); Md Fahim Anjum, Iowa City, IA (US); Raghuraman Mudumbai, Iowa City, IA (US)

(21) Appl. No.: 17/020,432

(22) Filed: Sep. 14, 2020

Related U.S. Application Data
(60) Provisional application No. 62/899,915, filed on Sep. 13, 2019.

(51) Int. Cl. A61B 5/04; A61B 5/0476; A61B 5/00; G16H 50/20; G16H 50/70; G16H 10/60; G16H 50/30; G16H 40/63

(52) U.S. Cl. CPC A61B 5/04012; A61B 5/0476; A61B 5/4082; A61B 5/7267; G16H 40/63; G16H 50/70; G16H 10/60; G16H 50/30; G16H 50/20

(57) ABSTRACT

The disclosed apparatus, systems and methods relate to diagnosing Parkinson's disease from electroencephalography (EEG) data. Embodiments herein have practical applications, including diagnosing Parkinson's disease. The methods and systems of the various implementations herein generate a diagnostic index which reflects the probability of the patient having Parkinson's disease. It uses a novel feature extraction method based on Linear Predictive Coding (LPC) which is used to extract Parkinson's disease related features from EEG recordings of the patient and a novel classification method based on Principal Component Analysis (PCA) is used to calculate the diagnostic index from these features.

### CROSS-REFERENCE TO RELATED APPLICATION(S)

This application claims the benefit under 35 U.S.C. § 119(e) to U.S. Provisional Application No. 62/899,915, filed Sep. 13, 2019 and entitled “Method and a System for Diagnosing Parkinson's Disease from Electroencephalography Data,” which is hereby incorporated herein by reference in its entirety.

### GOVERNMENT SUPPORT

This invention was made with Government support under Grant No. R01NS100849-01A1, awarded by the National Science Foundation. The government has certain rights in the invention.

### TECHNICAL FIELD

The various embodiments herein relate to the field of Neurology. More particularly, the implementations relate to the diagnosis of Parkinson's Disease using Electroencephalography (“EEG”).

### BACKGROUND

Parkinson's disease (“PD”) is a common progressive neurodegenerative disorder caused by the death of the dopamine-containing cells of the substantia nigra resulting in bradykinesia (i.e. slowness of movement), rigidity, tremor, and postural instability as well as non-moor symptoms. It has 0.3% estimated overall global prevalence which increases to more than 3% among people with more than 80 years of age. Between 2005 and 2030, the total number of people suffering from PD over age 50 is expected to double.

While the correct diagnosis of PD is crucial from both prognostic and therapeutic perspective, the accuracy of the diagnosis of PD is largely clinical and has not significantly improved in the last 25 years. Indeed, in a meta-analysis of 20 selected studies, pooled clinical diagnostic accuracy was 80.6% while the accuracy of clinical diagnosis by non-experts was 73.8%. An accurate diagnosis can be critical in selecting patients for advanced therapies such as adaptive brain stimulation (“aDBS”).

There is a need in the art for an improved system and method for diagnosing Parkinson's Disease.

### BRIEF SUMMARY

Discussed herein are various devices, systems and methods relating to diagnosing Parkinson's disease in human patients using EEG recordings. The input to the system is EEG data with multiple channels recorded from the scalp of the patient. The system output is a diagnostic index Linear-predictive-coding Electroencephalogy Algorithm in PD (“LEAPD”) which apart from providing a diagnosis also reflects the level of confidence in the diagnosis.

In the various Examples described herein, a system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. Other embodiments of this aspect include corresponding computer systems, apparatus, and computer programs recorded on one or more computer storage devices, each configured to perform the actions of the methods.

When software is used, any suitable programming, scripting, or other type of language or combinations of languages may be used to implement the teachings contained herein. However, software need not be used exclusively, or at all. For example, some embodiments of the methods and systems set forth herein may also be implemented by hard-wired logic or other circuitry, including but not limited to application-specific circuits. Combinations of computer-executed software and hard-wired logic or other circuitry may be suitable as well.

In Example 1, a method for diagnosing Parkinson's Disease (PD) from electroencephalography (EEG) data comprising: utilizing a system comprising: a computer processor for processing data, and a storage system for storing data on a storage medium, receiving an electroencephalography (“EEG”) time series data for diagnosis, calculating a Linear-predictive-coding Electroencephalogy Algorithm in PD (“LEAPD”) index for the EEG time series data, and diagnosing a patient from the EEG time series data using the LEAPD index.

In Example 2, the method of Example 1, wherein the calculating the LEAPD index for the EEG time series data comprises: filtering said EEG time series data with predetermined filter range, determining Linear Predictive Coding (LPC) coefficients from the EEG time series data with predetermined order and creating feature vector a, calculating a_PD and a_H from equations (21) and (22), calculating the distance vector D_PD from PD Principal Components Array (“PDPCA”) using equation (23), calculating the distance vector D_H from Healthy Principal Components Array (“HPCA”) using equation (24), and calculating LEAPD index p using equation (25).

In Example 3, the method of Example 1, wherein the diagnosing the patient from the EEG time series data using the LEAPD index comprises: generating Linear Predictive Coding (“LPC”) coefficients from the EEG time series data, recognizing that vector of LPC coefficients for PD patients and healthy controls lie in separate hyperplanes, finding the hyperplane for the PD patients and the hyperplane for the healthy controls, calculating the distances between the vector created by the LPC coefficients and the PD patients hyperplane and the healthy controls hyperplane, and determining whether the distance between the vector created by the LPC coefficients and the hyperplane for the PD patients is smaller than the distance between the vector created by the LPC coefficients and the hyperplane for the healthy controls, and if so, then diagnosing the patient as having PD.

In Example 4, the method of Example 1, wherein the diagnosing the patient from the EEG time series data using said LEAPD index comprises: diagnosing the patient as healthy if the LEAPD index value is less than 0.5, and diagnosing the patient as having PD if the LEAPD index value is greater than 0.5.

In Example 5, the method of Example 1, further comprising: utilizing a training dataset of multiple pre-diagnosed EEG time series data and a predetermined value of filter range, Linear Predictive Coding (“LPC”) order and number of components, filtering all EEG time series data of said training dataset with the predetermined filter range, calculating feature vector by determining LPC coefficients for each EEG time series data of said training dataset using Burg's method with the predetermined order, creating X_PD by combining the feature vectors of all EEG time series data from said training set pre-diagnosed as PD by using equation (9), determining PD Mean Array (“PDMA”) by using equation (11), determining a set of principal components from X_PD for PD, finding PD Principal Components Array (“PDPCA”) by taking the predetermined number of components from the set of principal components, creating X_H by combining the LPC coefficients of all EEG time series data from said training set pre-diagnosed as healthy by using equation (10), determining Healthy Mean Array (“HMA”) by using equation (12), determining a set of principal components from X_H for healthy, and finding Healthy Principal Components Array (“HPCA”) by taking the predetermined number of components from the set of principal components from X_H for healthy.

In Example 6, a system for diagnosing Parkinson's Disease (“PD”) from electroencephalography (EEG) data comprising: a computer processing system comprising: a processor, a storage medium associated with the processor, hardware associated with the processor, the hardware configured to receive EEG time series data for diagnosis, a software module configured to calculate a Linear-predictive-coding Electroencephalogy Algorithm in PD (“LEAPD”) index for the EEG time series data, and a software module configured to diagnose a patient from the EEG time series data using said LEAPD index.

In Example 7, the system of Example 6, wherein the software module configured to calculate the LEAPD index comprises: a filtering step of the EEG time series data with predetermined filter range, a Burg's method step for determining LPC coefficients from said EEG time series data with predetermined order and creating feature vector a, a step for calculating a_PD and a_H from equation (21) and equation (22), a step for calculating the distance vector D_PD from PD Principal Components Array (“PDPCA”) using equation (23), a step for calculating the distance vector D_H from Healthy Principal Components Array (“HPCA”) using equation (24), and a step for calculating LEAPD index p using equation (25).

In Example 8, the system of Example 6, wherein the software module configured to diagnose the patient from the EEG time series data using said LEAPD index comprises: a Linear Predictive Coding (“LPC”) coefficients generating step from the EEG time series data, a step of recognizing that vector of LPC coefficients for PD patients and healthy controls lie in separate hyperplanes, a step of finding the hyperplane for the PD patients and the hyperplane for the healthy controls, a step of calculating the distances between the vector created by the LPC coefficients and the hyperplane for the PD patients and the hyperplane for the healthy controls, and a step of determining whether the distance between the vector created by the LPC coefficients and the hyperplane for the PD patients is smaller than the distance between the vector created by the LPC coefficients and the hyperplane for the healthy controls, and if so, then a step of diagnosing the patient as having PD.

In Example 9, the system of Example 6, wherein the software module configured to diagnose the patient from the EEG time series data using said LEAPD index comprises: a step of diagnosing the patient as healthy if the LEAPD index value is less than 0.5, and a step of diagnosing the patient as having PD if the LEAPD index value is greater than 0.5.

In Example 10, the system of Example 6, further comprising: a training dataset of multiple pre-diagnosed EEG time series data, a predetermined value of filter range, Linear Predictive Coding (LPC) order and number of components, a step of filtering all EEG time series data of said training dataset with the predetermined filter range, a step of calculating feature vector by determining LPC coefficients for each EEG time series data of said training dataset using Burg's method with the predetermined order, a step of creating X_PD by combining the feature vectors of all EEG time series data from said training set pre-diagnosed as PD by using equation (9), a step of determining PD Mean Array (“PDMA”) by using equation (11) a step of determining a set of principal components from X_PD for PD, a step for finding PD Principal Components Array (“PDPCA”) by taking the predetermined number of components from the set of principal components, a step of creating X_H by combining the LPC coefficients of all EEG time series data from said training set pre-diagnosed as healthy by using equation (10), a step of determining Healthy Mean Array (“HMA”) by using equation (12), a step of determining a set of principal components from X_H for healthy, and a step of finding Healthy Principal Components Array (“HPCA”) by taking the predetermined number of components from the set of principal components from X_H for healthy.

In Example 11, a PD EEG system comprising a computer processor for processing data, and a storage system for storing data on a storage medium, wherein the processor and storage system are configured for receiving an EEG time series data, calculating a LEAPD index for the EEG time series data.

In Example 12, the method of claim 11, wherein the calculating the LEAPD index for the EEG time series data comprises filtering said EEG time series data with predetermined filter range.

In Example 13, the method of claim 11, wherein the calculating the LEAPD index for the EEG time series data comprises determining LPC coefficients from the EEG time series data with predetermined order and creating feature vector a.

In Example 14, the method of claim 11, wherein the calculating the LEAPD index for the EEG time series data comprises calculating a_PD and a_H from equations (21) and (22).

In Example 15, the method of claim 14, wherein the calculating the LEAPD index for the EEG time series data comprises calculating the distance vector D_PD from PDPCA using equation (23).

In Example 16, the method of claim 15, wherein the calculating the LEAPD index for the EEG time series data comprises calculating the distance vector D_H from HPCA using equation (24).

In Example 17, the method of claim 11, wherein the calculating the LEAPD index for the EEG time series data comprises calculating LEAPD index p using equation (25).

While multiple embodiments are disclosed, still other embodiments of the disclosure will become apparent to those skilled in the art from the following detailed description, which shows and describes illustrative embodiments of the disclosed apparatus, systems and methods. As will be realized, the disclosed apparatus, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1A illustrates a computer system for implementing the diagnostic system and/or method herein, according to one embodiment.

FIG. 1B depicts a primary block diagram of the overall system, according to one embodiment.

FIG. 2 depicts further details of the overall system, according to one embodiment.

FIG. 3 depicts further details of the Parameter selector Module of the FIG. 2 system.

FIG. 4 depicts further details of the Single Channel Parameter Selector Module of the FIG. 3 system.

FIG. 5 depicts further details of the Cross Validation Module of the FIG. 4 system.

FIG. 6 depicts further details of a Classifier Data Generator Module used in the FIG. 5 system and the FIG. 13 system.

FIG. 7 depicts further details of the Principal Component Selector Module of the FIG. 6 system.

FIG. 8 illustrates the Noise Filter Algorithm used in the FIG. 0.7 system and FIG. 0.10 system.

FIG. 9 depicts further details of the Principal Component Calculation Algorithm used in the FIG. 7 system.

FIG. 10 depicts further details of a Classifier Module used in the implementations of FIG. 5 and FIG. 14.

FIG. 11 depicts further details of the Classification Algorithm used in the FIG. 10 system.

FIG. 12 depicts further details of the Cross Validation Fold Generator Module of the FIG. 5 system.

FIG. 13 illustrates the Final Classification Data Generator Module of the FIG. 2 system.

FIG. 14 illustrates the Final Classification Method Module of the FIG. 0.2 system.

FIG. 15 illustrates the accuracy performance comparison of 62 channels for the best parameter selection of the FIG. 3 system.

FIG. 16A illustrates a diagnostic method according to one embodiment in comparison to the methods of the prior art in receiver-operating characteristics (“ROC”) curves.

FIG. 16B illustrates accuracy comparison of the diagnostic method according to one embodiment with the methods of the prior art in LOOCV.

FIG. 16C illustrates the output comparison of the diagnostic method according to one embodiment with respect to the methods of the prior art in box plots.

FIG. 16D illustrates the efficiency of the diagnostic method according to one embodiment in comparison to the methods of the prior art using total computational operations.

FIG. 17A illustrates the robustness of the diagnostic method according to one embodiment in various cross validations with receiver-operating characteristics (ROC) curves.

FIG. 17B illustrates accuracy comparison of the diagnostic method according to one embodiment in different cross validations.

FIG. 17C illustrates the output comparison of the diagnostic method according to one embodiment in various cross validations with box plots.

FIG. 18A illustrates the robustness of the diagnostic method according to one embodiment with ON and OFF medication states.

FIG. 18B illustrates accuracy comparison of the diagnostic method according to one embodiment for PD detection with or without medication.

FIG. 18C illustrates the correlation of the output of the diagnostic method according to one embodiment for with and without medication states.

FIG. 19A illustrates the electrodes of an EEG recording apparatus.

FIG. 19B illustrates the electrode positions of an EEG recording apparatus.

FIG. 19C illustrates a multi-channel EEG recording of a subject.

### DETAILED DESCRIPTION

The various embodiments disclosed or contemplated herein relate to an automated system for diagnosing Parkinson's Disease in human patients using EEG recordings. More specifically, the implementations herein relate to a novel method and system that provides a quantitative diagnostic index for diagnosing PD (called the Linear-predictive-coding EEG Algorithm for PD (LEAPD) index). In certain embodiments, the system provides for diagnosing PD from less than five minutes of EEG data. This index also provides a level of confidence in the diagnosis and outperforms all competing systems for diagnosing PD from qEEG.

The input to the system is EEG data with multiple channels recorded from the scalp of the patient. The system output is a diagnostic index Linear-predictive-coding Electroencephalogy Algorithm in PD (LEAPD) which apart from providing a diagnosis also reflects the level of confidence in the diagnosis. Electroencephalography (EEG) is a non-invasive technique that records the electric field produced by the neural activity in the brain reflecting the functional state of cortical layers and corresponding subcortical driving structures. Cortical structures can be abnormal in PD, and prior studies have extensively documented EEG abnormalities associated with PD in humans in animal models. Quantitative EEG (qEEG) analysis can be used to characterize potential markers of neuropsychiatric disorders including Alzheimer's disease, schizophrenia, major depressive disorder, and can be applied to PD.

According to certain embodiments, the method described herein has two phases: training (or “configuration”) phase and classification phase. Configuration phase uses a training dataset that consists of a set of pre-classified EEG data from PD and control subjects. The classification phase takes unclassified EEG data from any given new subject and generates the LEAPD index. This index takes values between 0 and 1. Being greater than 0.5 signifies PD; Being less than 0.5, healthy control. A value near 0.5 indicates a low level of confidence in the diagnosis.

All EEG data are first normalized to unit energy. Specifically, suppose an EEG time series is:

[e_{raw}(0),e_{raw}(1), . . . e_{raw}(N−1)], (1)

where the arguments represent sample indices. Then, the total energy of the time series is,

$$E = {\sum }_{i = 0}^{N - 1} {{e}_{raw} ( i )}^{2} \qquad ( 2 )$$

Now, the normalized time series is calculated as follows:

$${e}_{normalized} = [ \frac{{e}_{raw} ( 0 )}{\sqrt{E}} , \frac{{e}_{raw} ( 1 )}{\sqrt{E}} , … \frac{{e}_{raw} ( N - 1 )}{\sqrt{E}} ] \qquad ( 3 )$$

After this, a Noise Filter Algorithm is used on the normalized EEG data where the noise spikes are removed. The algorithm calculates the Fourier transform of the EEG time series data. Then, it replaces the Spectrum coefficients corresponding to the filter range with the spectrum coefficients from the nearby spectrums.

After this, the normalized and noise-removed EEG data goes through a zero-phase 6th Order Butterworth band-pass filter. The cutoffs of this band-pass filter are determined in the configuration phase.

The diagnostic system according to various implementations uses a novel feature extraction method based on Linear Predictive Coding (LPC) which is used to extract PD-related features from EEG recordings of the patient. These features are used in a novel classification method based on Principal Component Analysis (PCA) to calculate the diagnostic index called LEAPD index.

Linear Predictive Coding can predict time series behavior with extensive applications in speech processing and coding, analysis of myoelectric signals. The various embodiments herein use the known, recently developed Burg's method which reduces spectral loss and provides better frequency resolution. It can be used to estimate EEG power spectrum of migraine, epileptic and PD subjects.

Fundamentally, LPC fits an AR model to a time series. For example, suppose one has an EEG time series with N samples,

[e(0),e(1), . . . e(N−1)] (4)

Then, in L-th order LPC model, e(n) is predicted by [symbol] (n) by the forward linear predictor using data window [e(n−L), e(n−L+1), . . . , e(n−1)] with

$$( n ) = - {\sum }_{i = 1}^{L} {a}_{i} e ( n - i ) . \qquad ( 5 )$$

where a₁; (i=1, 2, . . . L) are called LPC coefficients. Similarly, e(n−L) is predicted by [symbol] (n−L) by the forward linear predictor using data window [e (n−L+1), . . . , e (n)] with

$$( n - L ) = - {\sum }_{i = 1}^{L} {a}_{i}^{*} e ( n - L + i ) . \qquad ( 6 )$$

The sum of the squares of the error between the original and estimated values for the forward linear prediction is,

$${F}_{L} = {\sum }_{n = L}^{N - 1} {\vert e ( n ) - ( n ) \vert}^{2} = {\sum }_{n = L}^{N - 1} {\vert e ( n ) + {\sum }_{i = 1}^{L} {a}_{i} e ( n - i ) \vert}^{2} . \qquad ( 7 )$$

Similarly, sum of the squares of the error for the backward linear prediction is,

$${B}_{L} = {\sum }_{n = L}^{N - 1} {\vert e ( n - L ) - ( n - L ) \vert}^{2} = {\sum }_{n = L}^{N - 1} {\vert e ( n - L ) + {\sum }_{i = 1}^{L} {a}_{i}^{*} e ( n - L + i ) \vert}^{2} . \qquad ( 8 )$$

Burg's algorithm minimizes the sum of the forward prediction error F_{L} and backward prediction error B_{L}. Given an EEG time series e(0), e(1), . . . e(N−1) with N samples, LPC of order L from Burg's algorithm generates L number of LPC coefficients, (a₁, a₂, . . . , a_{L}).

The feature vector is created using the LPC coefficients (a=[a₁, a₂ . . . a_{L}]^{T}) which is used for classification. This feature vector of LPC coefficients contains the information of the shape of the power spectral density (PSD) of the time series. The feature vector created from LPC coefficients captures the shape information of the entire PSD of EEG data. So, to capture the shape of the relevant part of the PSD, the EEG data go through an additional filter before calculating the LPC coefficients. In certain implementations, the classification method utilizes the fact that the feature vectors of LPC coefficients from PD subjects and the feature vectors of LPC coefficients from healthy subjects fall on two distinct hyperplanes. These hyperplanes can be identified via PCA by dimensionality reduction. PCA is a standard tool in modern data analysis and finds the directions in which the variance is maximized in multi-dimensional data sets. These directions are called Principal components.

In the training phase, according to one embodiment, a set of feature vectors from PD subjects and another set of feature vectors from healthy subjects are used to calculate the respective Principal components. Suppose, X_{PD} is a set of feature vectors from K number of PD subjects where i^{th} row of X_{PD} is the feature vector a_{PD}_{ᵢ} for i^{th} PD subject and X_{H} is a set of feature vectors from K number of healthy subjects where i^{th} row of X_{H} is the feature vector a_{H}_{ᵢ} for i^{th} healthy subject. Let the LPC order is L and also the number of Principal components to find is n. The dimension of X_{PD} and X_{H} will be K×L. So,

X_{PD}=[a_{PD}_{₁},a_{PD}_{₂}, . . . a_{PD}_{_{K}}]^{T} (9)

And

X_{H}=[a_{H}_{₁},a_{H}_{₂}, . . . a_{H}_{_{K}}]^{T} (10)

Let m_{PD} be the bias vector of X_{PD}

m_{PD}=[p₁,p₂, . . . p_{L}] (11)

where each element p, is the mean of the i^{th} column of array X_{PD}. Also, let m_{H} be the bias vector of X_{H}

m_{H}=[h₁,h₂, . . . h_{L}] (12)

where each element hᵢ is a mean of the i^{th} column of array X_{PD}. The dimension of m_{PD} and m_{H} will be 1× L

Now, a diagonal matrix D_{PD} is created using the elements of m_{PD} with the following equation:

$${D}_{P D} = [ \begin{matrix}{p}_{1} & … & 0 \\ & ⋱ & \\ 0 & … & {p}_{L}\end{matrix} ] \qquad ( 13 )$$

Similarly, a diagonal matrix D_{H} is created using the elements of m_{H} with the following equation:

$${D}_{H} = [ \begin{matrix}{h}_{1} & … & 0 \\ & ⋱ & \\ 0 & … & {h}_{L}\end{matrix} ] \qquad ( 14 )$$

Now the unbiased feature vector set X′_{PD} and X′_{H} is calculated using the following equations:

X′_{PD}=X_{PD}−(D_{PD}Q)^{T} (15)

X′_{H}=X_{H}−(D_{H}Q)^{T} (16)

Where Q is a matrix with L rows and K columns where each element is the number 1.

Next, matrix Y_{PD} and Y_{H} is calculated by scaling the unbiased feature vector set X′_{PD} and X′_{H} using the following equations:

$$\begin{matrix}{Y}_{P D} = \frac{{X}_{PD}^{′}}{\sqrt{K - 1}} & ( 17 ) \\ {Y}_{H} = \frac{{X}_{H}^{′}}{\sqrt{K - 1}} & ( 18 )\end{matrix}$$

After obtaining Y_{PD} and Y_{H}, singular value decomposition is performed and the matrices are factorized into the following form:

Y_{PD}=U_{P}Σ_{P}P^{T} (19)

Y_{H}=U_{C}Σ_{C}C^{T} (20)

Here the matrices U_{P}, P, U_{C}, C are orthogonal matrices and Σ_{P} and Σ_{C} are diagonal matrices whose diagonal elements are in decreasing order. The orthonormal basis vectors for the n dimensional hyperplane for PD are p₁, . . . , pₙ, the first n columns of P, and that for Healthy are h₁, . . . , hₙ, the first n columns of C. So, the i^{th} principal component of the PD subjects of the training phase is pᵢ and the i^{th} principal component of the healthy subjects of the training phase is hᵢ. After the two sets of Principal components are obtained, the system is capable of classifying any new subject.

As mentioned earlier, PCA is a data-dimensionality reduction algorithm that is not suitable for the direct application of classification. Hence, for the classification method, the various implementations herein use a novel algorithm based on vector projection which utilizes the principal components to classify new data. Suppose, for example, that the LPC feature vector of the given subject is a. First, mean of the PD dataset, denoted as m_{PD}, is subtracted from a and defined as a column vector named a_{PD}.

a_{PD}=a−m_{PD} (21)

Then, the mean of the healthy dataset, denoted as m_{H}, is subtracted from a and defined as a column vector named a_{H}.

a_{H}=a−m_{H} (22)

Now, vector projection formula is used to determine the minimum distance D_{PD} between the vector a_{PD} and the hyperplane created by the set of first n principal components associated with PD subjects. The distance vector D_{PD} can be calculated as follows:

$${D}_{P D} = {‖ {a}_{P D} - {\sum }_{k = 1}^{n} \frac{{p}_{k}^{T} {a}_{P D}}{{p}_{k}^{T} {p}_{k}} {p}_{k} ‖}_{2} \qquad ( 23 )$$

Similarly, the minimum distance D_{H} between the vector a_{H} and the hyperplane created by the first n number of principal components associated to healthy subjects is calculated as follows:

$${D}_{H} = {‖ {a}_{H} - {\sum }_{k = 1}^{n} \frac{{h}_{k}^{T} {a}_{H}}{{h}_{k}^{T} {h}_{k}} {h}_{k} ‖}_{2} \qquad ( 24 )$$

The LEAPD index calculator calculates the LEAPD index which is the diagnostic index for PD, according to certain embodiments. The LEAPD index (ρ) is calculated using the following equation:

$$0 \leq ρ = \frac{{D}_{H}}{{D}_{H} + {D}_{P D}} < 1 \qquad ( 25 )$$

When ρ<0.5, it is understood that the vector a is closer to the healthy subspace and if ρ>0.5, then it is closer to the PD subspace. ρ<0.5 corresponds to healthy controls and the opposite corresponds to PD. Hence, p acts as a measure of confidence in Parkinson's diagnosis. ρ value close to 0.5 indicated less confidence than values near 0 and 1.

In the configuration phase, first, the suitable cutoffs of the Butterworth filter and the optimum values of the LPC order L and the number of Principal Components n are determined. This is done by implementing an exhaustive search on the possible parameter space with some given parameter limits and comparing the performances of the combinations of parameters on a Training dataset using 10 fold cross validation method. The metric used for comparing performances is accuracy rate. Suppose the total PD subjects classified as PD is N_{PD}, the total healthy subjects classified as healthy is N_{Healthy} and total number of subjects is Nₜₒₜₐₗ. Then the accuracy rate is calculated using the following equation:

$$Accuracy rate = \frac{{N}_{P D} + {N}_{H e a l t h y}}{{N}_{t o t a l}} \times 100 \qquad ( 26 )$$

When optimum values of these parameters are obtained, the set of first n principal components associated with PD subjects (p₁, . . . , pₙ) are calculated using the Training dataset and combined in an array defined as Parkinson's Disease Principal Components Array (PDPCA) which is denoted as M_{PD}.

M_{PD}=[p₁, . . . ,pₙ] (27)

Similarly, the set of first n principal components associated to healthy subjects (h₁, . . . , hₙ) are calculated using the Training dataset and combined in an array defined as Healthy Principal Components Array (HPCA) which is denoted as M_{H}.

M_{H}=[h₁, . . . ,hₙ] (28)

Also, m_{PD} and m_{H} are calculated which can also be described as Parkinson's Disease Mean Array (PDMA) and Healthy Mean Array (HMA) respectively.

In the classification phase, for classifying a new subject, LEAPD index is calculated using equations (21), (22), (23), (24) and (25), as set forth above.

The description given above considers a single channel EEG data. However, it is understood that most known EEG recording devices are able to capture multi-channel data. To take advantage of the multi-channel EEG data, the system according to the various implementations herein can also take channel as a parameter and calculates LEAPD indices for multiple suitable channels. The suitable channels are selected based on their performances on the Training dataset. Suppose, the suitable channels are ch₁, ch₂, . . . ch_{T} and their respective LEAPD indices are ρ_{ch}_{₁}, ρ_{ch}_{₂}, . . . , ρ_{ch}_{_{T}}. Then, the final LEAPD index can be calculated by:

$${ρ}_{f i n a l} = \sqrt[T]{{\prod }_{i = 1}^{T} {ρ}_{c {h}_{i}}} \qquad ( 29 )$$

To summarize, the various embodiments herein relate to a system that can diagnose Parkinson's disease by analyzing EEG recordings of a human subject. The system requires pre-classified EEG recordings of healthy subjects and patients with Parkinson's disease for initial training and parameter selection. In certain embodiments, one unique feature is the fact that linear predictive coding (LPC) of EEG time series yields a vector of autoregressive parameters that lie on separate hyperplanes for control and PD patients. These can then be separated by using Principal Component Analysis (PCA). The LEAPD index quantifies this separation. There are many ways in which such a separation can be achieved, but, according to various embodiments herein, such a separation can be manifest between the EEG of PD and control.

### Definitions

Singular value decomposition (SVD): Singular value decomposition takes a matrix A and factorizes it into the following:

A=USV^{T}

Where UU^{T}=U^{T}U=1, VV^{T}=V^{T}V=1 and S is the diagonal matrix of the singular values of A. Columns of V called the right singular vectors.

Fourier transform: Fourier transform refers to the function which takes a time series and outputs the frequency domain representation of the time series.

Inverse Fourier transform: Inverse Fourier transform refers to the function which the frequency domain representation of a time series and outputs the time domain representation which is the original time series.

Independent component analysis: Independent component analysis is a process that decomposes a multivariate signal into independent non-Gaussian signals.

Linear Predictive Coding (LPC): Linear predictive coding fits an Auto-regressive model to a time series.

Leave-one-out cross validation (LOOCV): Leave one out cross validation is a specific type of cross validation where each subject of a dataset is tested using the rest of the dataset as training set and this is repeated for all subjects.

k-fold cross validation: k-fold cross validation is a specific type of cross validation where the dataset is randomly split into k number of subsets or folds with approximately equal sizes and each fold is tested using the rest of the folds combined as the training set. This is repeated for all of the k folds.

Power spectral density (PSD): Power spectral density is a function that represents the distribution of power into frequency components of a given time series data.

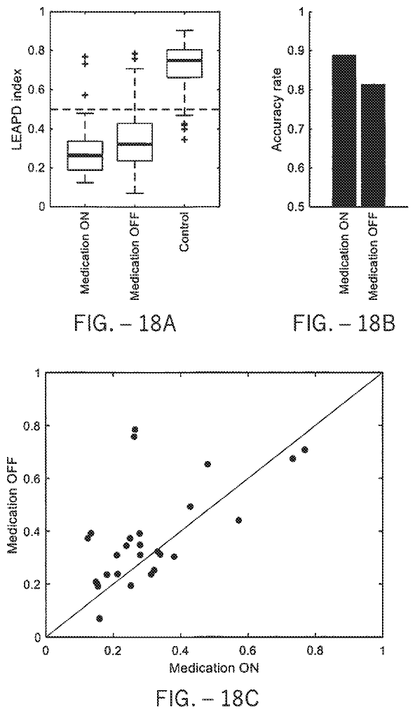

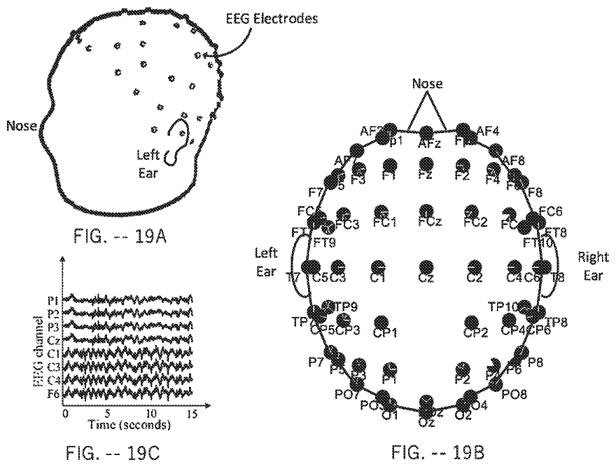

The input of the system and method according to the various embodiments herein is multi-channel EEG data from a human using an EEG recording apparatus. Typically, one such EEG recording apparatus, as known in the art, contains an array of EEG electrodes that are placed on the scalp of the human as depicted in FIG. 19A. Each electrode is placed on a specific position which is known in the art and records electrical activity of the brain from that position in a time series as depicted in FIG. 19B. These electrodes have specific names that are known in the art as channels and the recorded time series are called channel data for that channel as depicted in FIG. 19C. The EEG recording apparatus in one embodiment is a 64 channel Brain Vision system as depicted in FIG. 19B.

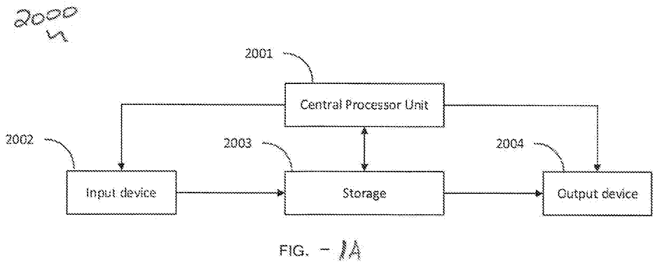

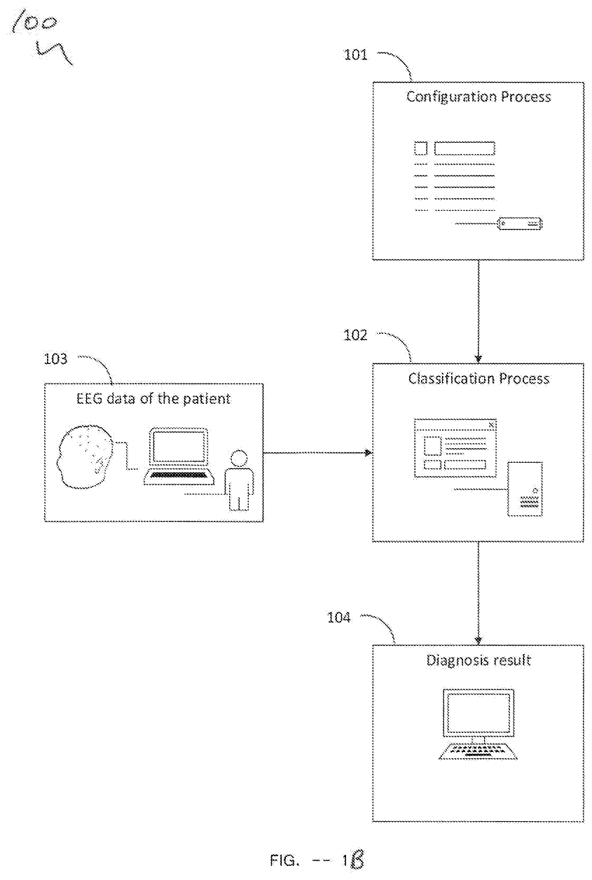

According to various embodiments, the system and method of the diagnostic system 100 can be carried out in various machines using numerous combinations of hardware and software. One such machine includes a computer system 2000, as known in the art, which is depicted in FIG. 1A. A computer system 2000 typically includes an Input device 2002 for receiving data and instructions, a Storage 2003 for storing instructions and computation results, an Output device 2004 for providing outputs of the instructions and computations and a Central Processing Unit (CPU) 2001 for executing numerous instructions and computations. The CPU 2001 also controls the Input device 2002, the Output device 2004 and the Storage 2003. The computer system 2000 in one embodiment includes a desktop computer containing program codes which cause the CPU 2001 to execute the system and method of the various implementations herein. The CPU 2001 can be a 64-bit processor, including a core i7 processor, manufactured by Intel Corporation. It is understood that any known computer system, including a processor on a network (such as a local area network or the Internet), can be used for carrying out the various system and method embodiments disclosed or contemplated herein.

FIG. 1B shows a primary block diagram of the overall diagnostis system 100, according to certain implementations. The diagnosis system 100 comprises a number of optional steps and/or sub-steps which can be performed in any order via the computer system 2000. In the implementation of the system 100 of FIG. 1, there are four steps: the configuration step (or process) 101, the classification step (or process) 102, the EEG data collection step 103, in which the EEG data of the patient is collected, and the generation of the diagnosis result 104.

The Configuration Process 101 utilizes a computer system (such as system 2000 as described above) that generates an output that contains parameter configurations essential for the Classification Process 102, as will be described in additional detail below. Typically, the configuration process 102 generates the output only once and passes it to the Classification Process 102.

The classification process 102 typically utilizes a computer system that receives EEG data as an input and generates a diagnostic index as output. It also takes the output from the Configuration Process 101 as input. Once it gets the output from Configuration Process 101, it stores the data in an internal storage and uses it for generating the diagnostic index unless it gets a new output from the Configuration Process 101. The diagnostic index is passed to the Diagnosis result 104.

Collection of EEG data from the patient 103 is typically the input portal of the system that takes multi-channel time-series EEG data of a patient as input and sends it as an output to the Classification Process 102. Typically, the input EEG data are recorded continuously from sintered Ag/AgCl electrodes across 0.1-100 Hz with a sampling rate 500 Hz on 64 channel Brain Vision system. Alternatively, any known EEG device can be used. The recorded EEG data is typically multi-channel data recorded on resting state of the patient. Typically, eye blinks are removed following independent component analysis, as known in the art, in the recorded EEG data before it is fed to the current system through EEG data of the patient 103 as input data.

The Diagnosis result generation step 104 displays the diagnostic index generated by the Classification Process 102. It receives the diagnostic index from the Classification Process 102 and displays whether the patient is PD or normal along with the diagnostic index. Typically, this diagnostic index has a range of 0 to 1, as will be described in detail below. This diagnostic index is hereafter defined as LEAPD index. It expresses the probability of the patient having Parkinson's disease.

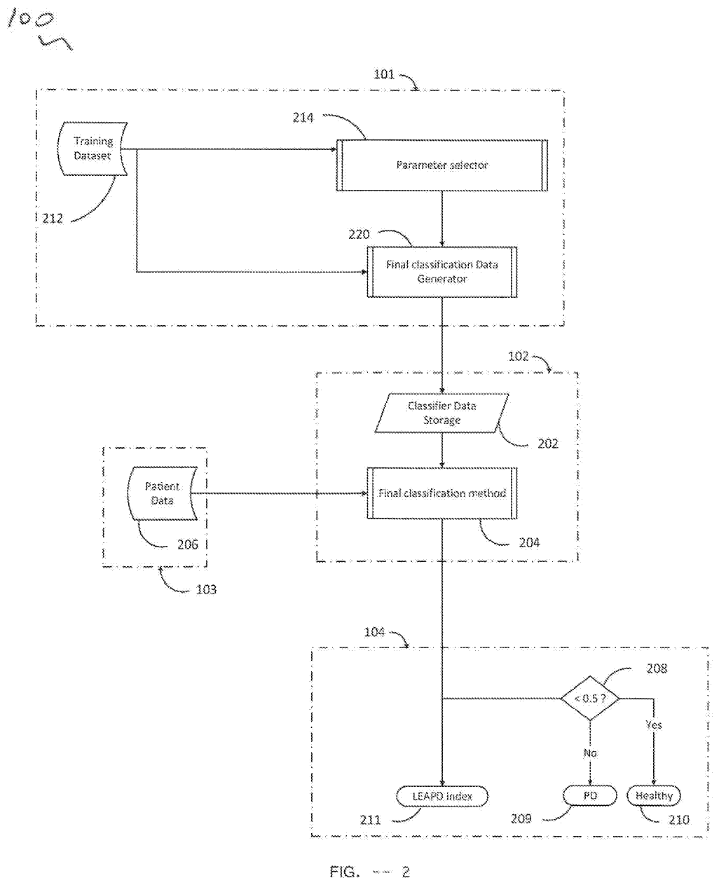

FIG. 2 shows a detailed block diagram of the overall system 100 with further details in comparison to FIG. 1B. The EEG data collected via the collection step 103 for classification enters the system 100 through Patient data 206 which contains the input EEG data which is typically multi-channel time-series data. This input goes into the Final classification method 204, which is part of the classification step 102.

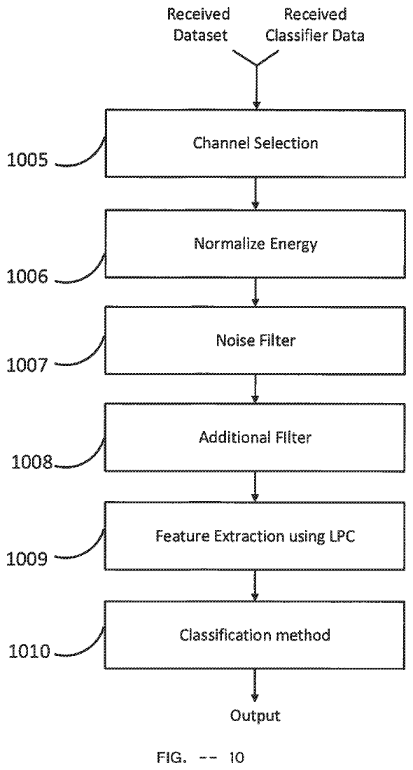

The Final classification method 204 can be, according to certain embodiments, performed via a Final classification method module such as the module depicted in FIG. 14. The final classification method 204 typically outputs a diagnostic index defined as the LEAPD index, as described in detail elsewhere herein. It takes the input EEG data from Patient data 206 as EEG Data. It also takes Classifier Dataset as input which is provided by the Classifier Data storage 202 (which is also part of the classification process 102). The Classifier Dataset is defined as a collection of Classifier Data, which can be, according to certain embodiments, a collection of a Channel, a Filter range, an LPC order, a PD Principal Components Array (PDPCA), a PD Mean Array (PDMA), a Healthy Principal Components Array (HPCA) and a Healthy Mean Array (HMA). Alternatively, the information included in the classifier data can be any known data that can be useful for purposes of classification.

The generation of the diagnosis result step 104 includes receiving the LEAPD index from the Final classification method 204 as an input and displaying the LEAPD index as an output. The LEAPD index from Final classification method 204 also goes into decision block 208, which outputs either a PD diagnosis 209 or Healthy diagnosis 210. These outputs are visible to the user of the system 100.

Returning to the classification process 102, the Classifier Data Storage 202 stores Classifier Dataset which are essential for the Final classification method 204. The inputs to this module are Classifier Dataset from the Final classification Data Generator 220 of the configuration process 101. The Classifier Data Storage 202 takes Classifier Dataset as input, stores the Classifier Dataset and provides the stored Classifier Dataset as output to the Final classification method 204. The data structure of the Classifier Data Storage 202 can, in certain embodiments, include Channel, Filter range, LPC order, PD Principal Components Array (PDPCA), PD Mean Array (PDMA), Healthy Principal Components Array (HPCA) and Healthy Mean Array (HMA). The data structure according to this embodiment is shown in the following table:

TABLE 1

| | | | | | | |
|---|---|---|---|---|---|---|
| Channel | Filter range | LPC Order | PDPCA | PDMA | HPCA | HMA |

Alternatively, any other known datapoints can be included in the data structure.

The Training Dataset 212 of the classification process 102 contains Pre-classified multi-channel EEG data from multiple healthy humans and multiple PD subjects.

Stored data in Training Dataset 212 typically includes classification (PD or healthy) and multi-channel EEG data. In one particular embodiment, Training Dataset 212 contains a pre-classified dataset of 27 EEG recordings with 62 channels from 27 PD patients and 27 EEG recordings with 62 channels from 27 PD healthy subjects. This stored data is hereafter defined as Training Dataset. As part of the configuration process 101, the Training Dataset 212 supplies the Training dataset to Parameter selector 214 and Final classification Data Generator 220.

The Parameter selector 214 of the configuration process 101 can be performed, according to certain embodiments, via a Parameter selector module such as the module set forth in additional detail in FIG. 3. It determines the best Channels and their respective values of Filter range, LPC Order and Number of components for classification. It takes the output dataset from Training dataset 212 as input and generates a Parameter Combination as output which is delivered to Final classification Data Generator 220. Parameter Combination is defined as a set where each element contains a Channel and its Corresponding Parameter. Parameter is defined as a collection of a Filter range, an LPC order and a number of components. The output structure is shown in the following table:

TABLE 2

| | |
|---|---|
| Channel | Parameter |

The structure of Parameter is shown in the following table:

TABLE 3

| | | |
|---|---|---|
| Filter range | LPC order | Number of components |

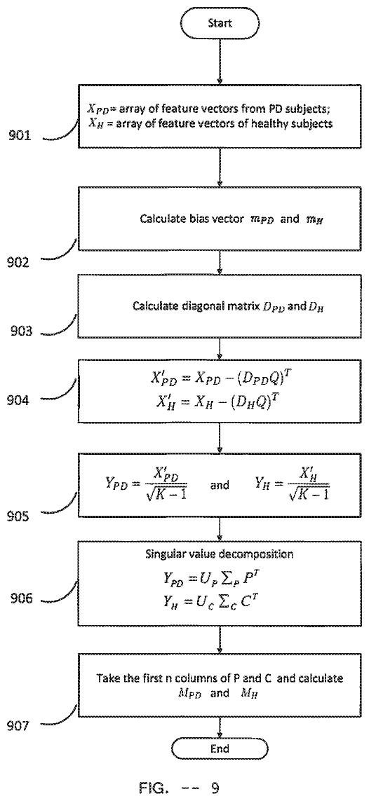

The Final classification Data generator 220 is a Final Classification Data Generator module of FIG. 13. It generates Classifier Dataset to be stored in the Classifier Data storage 202. The Final classification Data generator 220 takes two inputs: Dataset and Parameter Combination. The Parameter Combination from Parameter selector 214 is delivered as Parameter Combination input and the Training dataset from Training Dataset 212 is delivered as Dataset input to the Final classification Data generator 220. The output of the Final classification data generator 220 is the Classifier Dataset which has the same structure as described in Table 1 and the output is delivered to Classifier data Storage 202.

Parameter selector Module—FIG. 3

In FIG. 3, a block diagram of the Parameter selector module is shown. It takes the Training dataset as Dataset input. It generates the Parameter Combination as output. The output as the same structure as shown in Table 2.

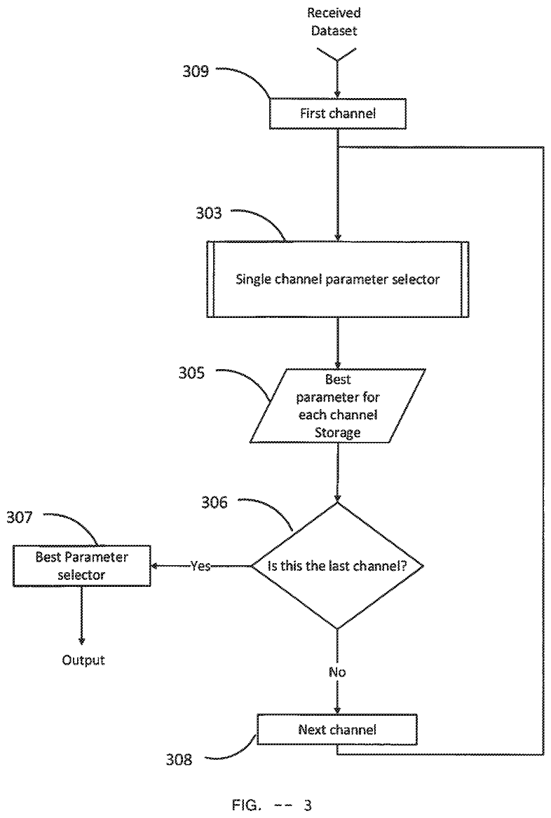

The First Channel 309 selects the first channel in the received Dataset, sets the first channel as current channel and outputs the current channel to Single channel parameter selector 303 which is the Single channel parameter selector module of FIG. 4. The Single channel parameter selector 303 has two inputs: Dataset and Channel. The Single channel parameter selector 303 takes the current channel as Channel input. It also takes the received Dataset as Dataset input. The Single channel parameter selector 303 typically calculates the best Parameter for the given channel and its corresponding accuracy rate performance (ACC) on the Training Dataset. The output of the single channel parameter selector 303 includes a particular Parameter and a corresponding ACC for the given channel which are delivered to Best parameter for each channel storage 305.

The Best parameter for each channel Storage 305 stores the output of the single channel parameter selector 303 for different channels. It takes the outputs of the single channel parameter selector 303 as input and stores the Parameter output and the corresponding ACC output along with the current channel. The data structure of this storage is shown in the following table:

TABLE 4

| | | |
|---|---|---|
| Channel | Parameter | ACC |

The decision block 306 checks whether current channel is the last channel of the received Dataset or not. If the current channel is not the last channel of the received Dataset, next channel is loaded by Next channel 308 which sets the loaded channel as the current channel and feeds it to the Single channel parameter selector 303. If the current channel is indeed the last channel of the received Dataset, Best Parameter selector 307 generates output for the Parameter Selector Module.

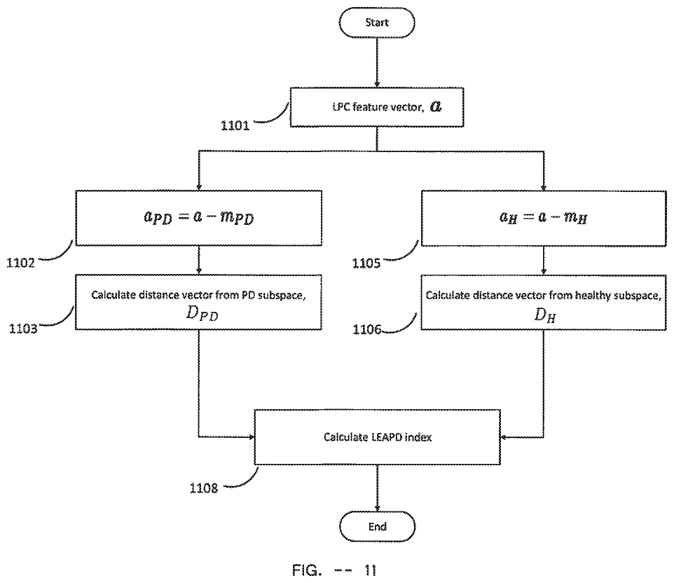

Best Parameter selector 307 sorts the data stored by the Best parameter for each channel Storage 305 in terms of the ACC value and selects the channels and their corresponding Parameter that have ACC values more than a threshold. Typically, this threshold value is set as 83%. In one particular embodiment, 62 channels are compared using ACC value which is illustrated in FIG. 15. It shows that ACC values for channel TP8, P6, FC5, PO8, O2, and CP5 are more than 83%. Hence, these channels and their corresponding Parameters are selected by Best Parameter selector 307. The output of Best Parameter selector 307 is a Parameter Combination which is created by the selected channels and their respective Parameter. This output is delivered as the output of the Parameter selector Module which has the same structure as shown in Table 2.

Single Channel Parameter Selector Module—FIG. 4

In FIG. 4, a block diagram of the single channel parameter selector module is shown. It takes a Channel input and a Dataset input. The Single channel parameter selector module delivers a Parameter for the given Channel and a corresponding ACC as output. The single channel parameter selector module typically includes a Parameter ranges storage 403. This parameter ranges storage 403 stores pre-selected upper and lower limits of the first and second part of the filter ranges and a pre-selected range of the LPC order. In one particular embodiment, parameter ranges storage stored 2.5 Hz as the upper and lower limits of the first part of the filter range, stored 8 Hz and 14 Hz as the upper and lower limits of the second part of the filter range respectively and stored 2 to 10 as the range of the LPC order.

The Combination generator 402 takes the limits from the Parameter ranges storage 403 and generates possible combinations of Filter range, LPC order and number of components within the limits. Each combination includes a particular Filter range, LPC order and number of components. Note that the number of components cannot exceed the LPC order. The Combination generator 402 typically generates the filter ranges by varying the first part and the second part of the filter range within the respective upper and lower range and for each filter range, it varies the LPC order from the minimum value to the maximum value. For each of the above cases of a particular filter range and LPC order, number of components is varied. Minimum value of number of components is typically 1 and the maximum value is typically obtained by subtracting 1 from the particular LPC order of that case. In one particular embodiment, given 2.5 Hz as the upper and lower limits of the first part of the filter range, 8 Hz and 9 Hz as the upper and lower limits of the second part of the filter range respectively and LPC order range of 2 to 4, the Combination generator 402 generates 12 possible combinations and the generated combinations of this particular embodiment are shown in Table 5.

TABLE 5

| Filter range | LPC order | Number of components |
|---|---|---|
| 2.5-8 Hz | 2 | 1 |
| 2.5-8 Hz | 3 | 1 |
| 2.5-8 Hz | 3 | 2 |
| 2.5-8 Hz | 4 | 1 |
| 2.5-8 Hz | 4 | 2 |
| 2.5-8 Hz | 4 | 3 |
| 2.5-9 Hz | 2 | 1 |
| 2.5-9 Hz | 3 | 1 |
| 2.5-9 Hz | 3 | 2 |
| 2.5-9 Hz | 4 | 1 |
| 2.5-9 Hz | 4 | 2 |
| 2.5-9 Hz | 4 | 3 |

When Combination generator 402 finishes generating all possible combinations, the first combination is set as the current Parameter and passed to the Cross Validation 408 as Parameter by First combination 405.

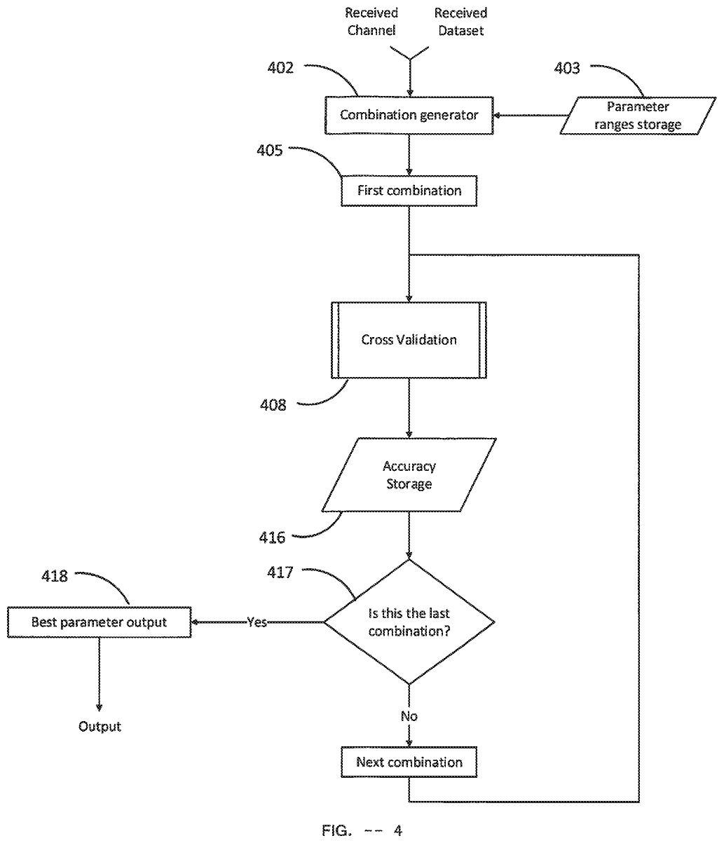

The Cross validation 408 is a Cross Validation module of FIG. 5. It takes three inputs: Dataset, Parameter and Channel. The current Parameter is delivered as Parameter, the Dataset received by the Single Channel Parameter Selector Module is delivered as Dataset and the Channel received by the Single Channel Parameter Selector Module is delivered as Channel. It performs k-fold Cross Validation on the Training dataset and calculates the ACC for the current parameter. The output of the Cross validation 408 is a ACC value which is delivered to the Accuracy Storage 416 as input.

The Accuracy Storage 416 stores ACC values of each Parameter. It takes the ACC value as input from the output of Cross validation 408 and stores it along with the current Parameter. The data structure of the Accuracy Storage 416 includes Parameter and ACC which is depicted in Table 6.

TABLE 6

| | |
|---|---|
| Parameter | ACC |

The decision block 417 checks whether the current combination is the last combination of the generated combinations by the Combination generator 402. If the current combination is not the last combination then the next combination is set as the current Parameter and passed to the Cross Validation 408. Otherwise, Best parameter output 418 finds the best performing Parameter and outputs the Parameter along with its corresponding ACC value.

The Best parameter output 418 compares takes all Parameter with their respective ACC values stored in the Accuracy Storage 416 and selects the Parameter with the maximum ACC value as the best Parameter. This best Parameter and its corresponding ACC value are delivered as outputs of the Single Channel Parameter Selector Module.

In FIG. 5, a block diagram of a Cross Validation Module is shown. The Cross Validation takes three inputs: Dataset, Parameter and Channel. It outputs an ACC value. Cross Validation Module typically includes a Fold value storage 501 which stores a pre-selected fold value. Fold value storage 501 delivers the stored fold value as output to Cross validation fold generator 502. In one embodiment, the pre-selected fold value stored in Fold value storage 501 is 10.

The Cross Validation Module starts at the Cross validation fold generator 502 which is a Cross Validation Fold Generator Module of FIG. 12. It takes the fold value as input from the Fold value storage 501. It also takes the Dataset received by the Cross Validation Module as input Dataset. The output of Cross validation fold generator 502 is a collection of data-folds. Data-fold is defined as a collection of two datasets of EEG data where one is named as Training set and the other one is named as Test set. When Cross validation fold generator 502 finishes generating the collection of data-folds and delivers them as output, First data-fold 517 takes the first data-fold of the generated collection of data-folds by Cross validation fold generator 502, sets it as the current data-fold and starts Classifier Data Generator 505.

The Classifier data generator 505 is a Classifier Data Generator Module of FIG. 6. It takes three inputs: Dataset, Parameter and Channel. The Training set of the current data-fold is delivered as Dataset. The Parameter received by the Cross Validation Module is delivered as Parameter and the Channel received by the Cross Validation Module is delivered as Channel. It creates a Classifier Data for the received Channel as output.

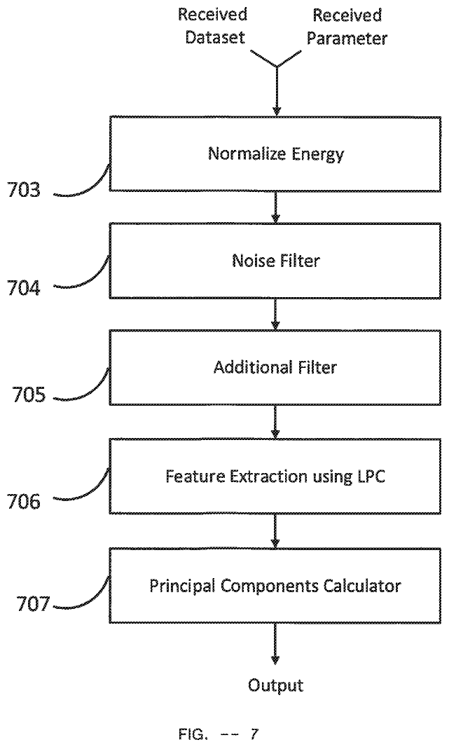

The Classifier 506 is a Classifier Module of FIG. 10. It takes two inputs: Dataset and Classifier Data. The Test set of the current data-fold is delivered as Dataset and the Classifier Data delivered by Classifier data generator 505 is delivered as Classifier Data. The Classifier 506 generates an array of LEAPD indices for the Test set of the current data-fold where each LEAPD index of the array corresponds to a particular EEG data of the Test set. The Classifier 50 delivers this array of LEAPD indices as output to the Accuracy Calculator 514.

The Accuracy calculator 514 takes the array of LEAPD indices for the Test set of the current data-fold from the Classifier 506 as input and calculates the accuracy rate. It delivers the accuracy rate as output to the Accuracy Storage 510. For each LEAPD index of the received array of LEAPD indices, Accuracy calculator 514 checks whether each LEAPD index is below 0.5. If true, then the EEG data corresponding to that particular LEAPD index is classified as Healthy otherwise it is classified as PD. After this, as the dataset is pre-classified, Accuracy calculator 514 compares the original classification of PD and healthy with the calculated classification from LEAPD indices and calculates the total number of correct PD classifications and total number of correct healthy classifications. Accuracy rate is calculated using (26) and delivered as ACC for the output.

The Accuracy Storage 510 takes the ACC value output from the Accuracy calculator 514 as input and stores ACC value for the current data-fold. Typically, the Accuracy Storage 510 stores incoming ACC values in an array. When a new ACC value comes as an input from the Accuracy calculator 514, Accuracy Storage 510 stores the new ACC value in this array ACC values.

The decision block 512 checks whether current data-fold is the last data-fold of the generated collection of data-folds by Cross validation fold generator 502. It the condition is false, the next data-fold of the generated collection of data-folds by Cross validation fold generator 502 is set as the current data-fold and Classifier Data Generator 505 is started. And if the condition is true, the Output generator 513 generates the output.

The output generator 513 takes the array of ACC values stored in the Accuracy Storage 510, calculates the average of the ACC values in the array and delivers the calculated average as ACC in the output.

FIG. 6 shows a block diagram of a Classifier Data Generator Module. It takes three inputs: Dataset, Channel and Parameter. It includes the Channel selection 604, the Principal component selector 609 and the Data Combiner 612.

Channel selection 604 takes the received Dataset and the received Channel as inputs. The received Dataset is typically consisting of multi-channel EEG data. From the received Dataset, it selects only the EEG data corresponding to the received Channel and creates a channel-selected EEG dataset with this selected EEG data. This channel-selected EEG dataset contains EEG data only for the received channel and it is delivered as Dataset output to the Principal component selector 609.

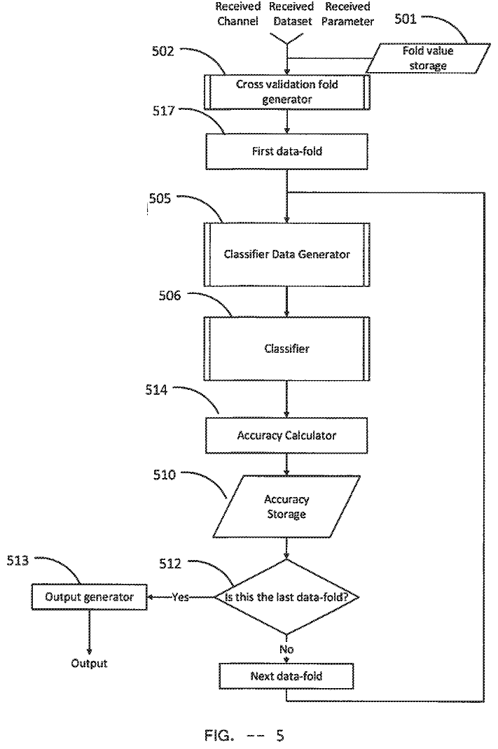

The Principal component selector 609 is a Principal Component Selector Module of FIG. 7. It takes two inputs: Dataset and Parameter. The channel-selected EEG dataset output from the Channel selection 604 is delivered as Dataset and the Parameter received by the Classifier Data Generator Module is delivered as Parameter. Principal component selector 609 generates a PD Principal Components Array (PDPCA), a PD Mean Array (PDMA), a Healthy Principal Components Array (HPCA) and a Healthy Mean Array (HMA) as outputs which are passed to the Data combiner 612.

The Data combiner 612 takes the PD Principal Components Array (PDPCA), the PD Mean Array (PDMA), the Healthy Principal Components Array (HPCA) and the Healthy Mean Array (HMA) as inputs from the Principal component selector 609. It combines these four arrays along with the Parameter received by the Classifier Data Generator Module and creates a Classifier Data for output. The output has the same structure as shown in Table 1.

Principal Component Selector Module—FIG. 7

FIG. 7 shows a block diagram of a Principal Component Selector Module. It takes two inputs: Dataset and Parameter. It generates a PD Principal Components Array (PDPCA), a PD Mean Array (PDMA), a Healthy Principal Components Array (HPCA) and a Healthy Mean Array (HMA) as outputs.

The Normalize energy 703 takes the given Dataset as input and normalize the total energy of the time series data of received Dataset using (2) and (3). This process is done for each EEG time series in the received Dataset. Then, the Normalized EEG dataset is passed to Noise Filter 704.

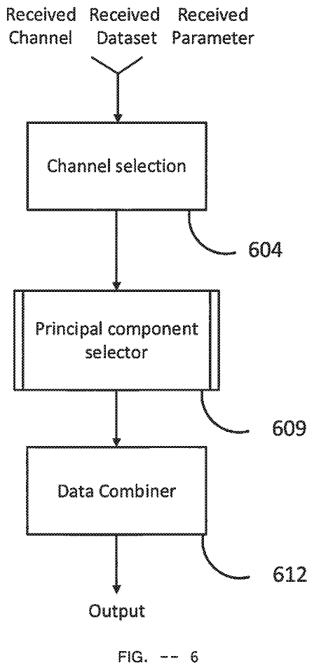

The noise Filter 704 takes the normalized EEG dataset and filters the noises. Typically, the noises occur in some specific frequency ranges. These frequency ranges are defined as noisy filter ranges. Typically, these filter ranges are pre-selected after manually inspecting the PSD of the time series EEG data. In one specific embodiment, the selected noisy filter ranges are: 55.6 Hz-63 Hz, 177 Hz-183 Hz and 197-203 Hz. The noise filter 704 takes each EEG time series and filters the noise for each of the noisy filter ranges by implementing the Noise Filter Algorithm of FIG. 8. After this process, the noise-filtered EEG dataset is passed to Additional Filter 705.

The Additional Filter 705 takes the noise-filtered EEG dataset as input and filters each of the EEG time series in the noise-filtered EEG dataset with a specific filter range. The specific filter range is determined from the Parameter received by the Principal Component Selector Module. Each of the EEG time series goes through a zero-phase 6th order Butterworth pass-band filter with frequency bands defined by the specific filter range. After this process, the filtered EEG dataset is passed to the Feature Extraction using LPC 706.

The Feature Extraction using LPC 706 takes the filtered EEG dataset from the Additional Filter 705 as input. It also takes LPC order as input from the Parameter received by the Principal Component Selector Module. It applies Burg's method to calculate LPC coefficients (a₁, a₂, . . . , a_{L}) from each of the EEG time series of the filtered EEG dataset from the Additional Filter 705. Specifically, given an EEG time series [e(0), e(1), . . . , e(N−1)] with N samples and denoting LPC coefficients of k-th order as a_{k,i}; i∈{1, 2, . . . , k}, forward prediction error f_{k,n} for predicting e(n) is,

$${f}_{k , n} = e ( n ) + {\sum }_{i = 1}^{k} {a}_{k , i} e ( n - i ) \qquad ( 30 )$$

And the backward prediction b_{k,n} for predicting e(n−k) is,

$${b}_{k , n} = e ( n - k ) + {\sum }_{i = 1}^{k} {a}_{k , i} e ( n - k + i ) \qquad ( 31 )$$

Then LPC coefficients of order L can be calculated as follows:
0. Initialization:

k=a_{0,0}=1 (32)
1. Calculate a_{k,k}:

$${a}_{k , k} = \frac{2 {Σ}_{n = k}^{N - 1} {f}_{k - 1 , n}^{*} {b}_{k - 1 , n - 1}}{{Σ}_{n = k}^{N - 1} ( {\vert {f}_{k - 1 , n} \vert}^{2} + {\vert {b}_{k - 1 , n - 1} \vert}^{2} )} \qquad ( 33 )$$

2. Calculate a_{k,i} for i∈{1, 2, . . . , k−1}:

a_{k,i}=a_{k-1,i}+a_{k,k}a*_{k-1,k-i} (34)
3. Update the prediction errors:

f_{k,n}=f_{k-1,n}+a_{k,k}b_{k-1,n-1} (35)

b_{k,n}=b_{k-1,n-1}+a*_{k,k}e_{k-1,n} (36)
4. Stopping condition: if k is equal to L, then go to step 7, otherwise go to step 5.
5. Increment iteration counter:

k=k+1 (37)
6. Back to step 1.
7. Finish the algorithm and output the LPC coefficients as (a₁, a₂, . . . , a_{L}) where aᵢ=a_{L,i}; i∈{1, 2, . . . , L}.

These LPC coefficients (a₁, a₂, . . . , a_{L}) are used to create a feature vector of the EEG time series (a=a₂ a_{L}F). This process is repeated for each of the EEG time series of the input EEG dataset. Feature vectors of all the EEG time series pre-classified as PD are combined in an array X_{PD} where each row is a vector of LPC coefficients of one EEG time series. Similarly, feature vectors of all the EEG time series pre-classified as healthy are combined in an array X_{H} where each row is a feature vector of one EEG time series. Lastly, X_{PD} and X_{H} are delivered to the Principal Components Calculator 707.

The Principal Components Calculator 707 takes the array of feature vectors X_{PD} and X_{H} from Feature Extraction using LPC 706. It also takes the number of components as input from the Parameter received by the Principal Component Selector Module. The Principal Components Calculator 707 implements the Principal Component Selector Algorithm of FIG. 9 and delivers PD Principal Components Array (PDPCA), PD Mean Array (PDMA), Healthy Principal Components Array (HPCA) and Healthy Mean Array (HMA) as outputs.

FIG. 8 shows a flow diagram of the Noise Filter Algorithm. The inputs to this algorithm are an EEG time series e(n) and a Filter range. The output of this algorithm is noise filtered EEG time series, e(n). Then, the Noise Filter Algorithm implements the following steps:
1. Calculate the Discrete Fourier transform of the given EEG time series Fₑ(f) from e(n) where f∈F so that F is the support of frequency variable f
2. Determine the subset of F which contains all elements of F that are within the received Filter range and store it as array β in increasing order such that β(1)<β(2)<β(3) etc.
3. Calculate Δ which is the total number of elements in β.
4. If Δ is as odd number, then execute steps 5 to 9 else execute steps 10 to 14
5. Calculate Δ₁ using the following equation:

$${Δ}_{1} = \frac{( Δ - 1 )}{2} \qquad ( 38 )$$

6. Determine β₁ which is defined in the following equation:

β₁=[β(1),β(2) . . . β(Δ₁)] (39)
7. Determine β₂ which is defined in the following equation:

β₂=[β(Δ₁×2),β(Δ₁+3) . . . β(Δ)] (40)
8. Determine \hat{F}ₑ(f) which is defined in the following equation:

$${\hat{F}}_{e} ( f ) = \begin{matrix} \\ \\ \end{matrix} \{ \begin{matrix}{F}_{e} ( f + β ( 1 ) - β ( {Δ}_{1} ) ) & if f ∈ {β}_{1} \\ {F}_{e} ( f - β ( 1 ) + β ( {Δ}_{1} ) ) & if f ∈ {β}_{2} \\ \frac{{F}_{e} ( β ( {Δ}_{1} ) ) + {F}_{e} ( β ( {Δ}_{1} + 2 ) )}{2} & if f = β ( {Δ}_{1} + 1 ) \\ {F}_{e} ( f ) & otherwise\end{matrix} \qquad ( 41 )$$

9. Determine ê(n) by applying inverse Discrete Fourier transform on \hat{F}ₑ(f) and deliver ê(n) as output.
10. Calculate Δ₁ using the following equation:

$${Δ}_{1} = \frac{Δ}{2} \qquad ( 42 )$$

11. Determine β₁ which is defined in the following equation:

β₁=[β(1),β(2) . . . β(Δ₁)] (43)
12. Determine β₂ which is defined in the following equation:

β₂=[β(Δ₁+1),β(Δ₁+2) . . . β(Δ)] (44)
13. Determine \hat{F}ₑ(f) which is defined in the following equation:

$${\hat{F}}_{e} ( f ) = \{ \begin{matrix}{F}_{e} ( f + β ( 1 ) - β ( {Δ}_{1} ) ) & if f ∈ {β}_{1} \\ {F}_{e} ( f - β ( 1 ) + β ( {Δ}_{1} ) ) & if f ∈ {β}_{2} \\ {F}_{e} ( f ) & otherwise\end{matrix} \qquad ( 45 )$$

14. Determine ê(n) by applying inverse Discrete Fourier transform on [symbol] (f) and deliver ê(n) as output.

FIG. 9 shows a flow diagram of the Principal Component Selector Algorithm. The inputs to this algorithm are the feature vector array X_{PD} and X_{H} and the number of components, n. It delivers PD Principal Components Array (PDPCA), PD Mean Array (PDMA), Healthy Principal Components Array (HPCA) and Healthy Mean Array (HMA) as outputs. The principal component selector algorithm centers the feature vector array by subtracting of each column from each column. Then the right singular vectors are calculated from feature vector array using SVD. Finally, selected columns of the right singular vectors and the array of columns are delivered as outputs. The steps of the principal component selector algorithm are the following:
1. Calculate bias vectors m_{PD} and m_{H} using (11) and (12)
2. Calculate matrix D_{PD} and D_{H} using (13) and (14)
3. Calculate unbiased feature vector set X′_{PD} and X′_{H} using (15) and (16)
4. Calculate Y_{PD} and Y_{H} using (17) and (18)
5. Perform singular vector decomposition on Y_{PD} and Y_{H} obtain right singular vector P and C from (19) and (20)
6. Take first n columns of P, p₁, . . . , pₙ, and calculate M_{PD} using (27)
7. Take first n columns of C, h₁, . . . , hₙ, and calculate M_{H} using (28)

After implementing the above steps, M_{PD} is delivered as PD Principal Components Array (PDPCA), M_{H} is delivered as Healthy Principal Components Array (HPCA), m_{PD} is delivered as PD Mean Array (PDMA) and m_{H} is delivered as Healthy Mean Array (HMA).

FIG. 10 shows a block diagram of a Classifier Module. It takes two inputs: Dataset and Classifier data. It generates an array of LEAPD indices for the received Dataset and delivers it as output.

The Channel Selection 1005 takes the received Dataset and the Channel from the received Classifier Data as inputs. Typically, the received Dataset consists of multi-channel EEG data. From the received Dataset, Channel Selection 1005 selects only the EEG data corresponding to the received Channel and creates a channel-selected EEG dataset with this selected EEG data. This channel-selected EEG dataset contains EEG data only for the received channel and it is delivered as Dataset output to the Normalize Energy 1006.

The Normalize energy 1006 takes the channel-selected EEG dataset as input from Channel Selection 1005 and normalizes the total energy of the time series data of the EEG dataset. The functionalities of the Normalize energy 1006 are identical to the Normalize Energy 703 of FIG. 7.

The Noise Filter 1007 takes the normalized EEG dataset from The Normalized energy 1006 and filters the noises. The functionalities of the Noise Filter 1006 are identical to the Noise Filter 704 of FIG. 7.

The Additional Filter 1008 takes the noise-filtered EEG dataset from the noise filter 1007 as input and filters each of the EEG time series with a specific filter range.

The specific filter range is determined from the Classifier Data received by the Classifier Module. The functionalities of the Additional Filter 1008 are identical to the Additional Filter 705 of FIG. 7.

The Feature Extraction using LPC 1009 takes the filtered EEG dataset from the Additional Filter 1008 as input and it also takes LPC order as input from the Classifier Data received by the Classifier Module. It uses Burg's method for LPC and generates array of feature vectors which is passed to the classification method 1010. The functionalities of the Feature Extraction using LPC 1009 are identical to the Feature Extraction using LPC 706 of FIG. 7.

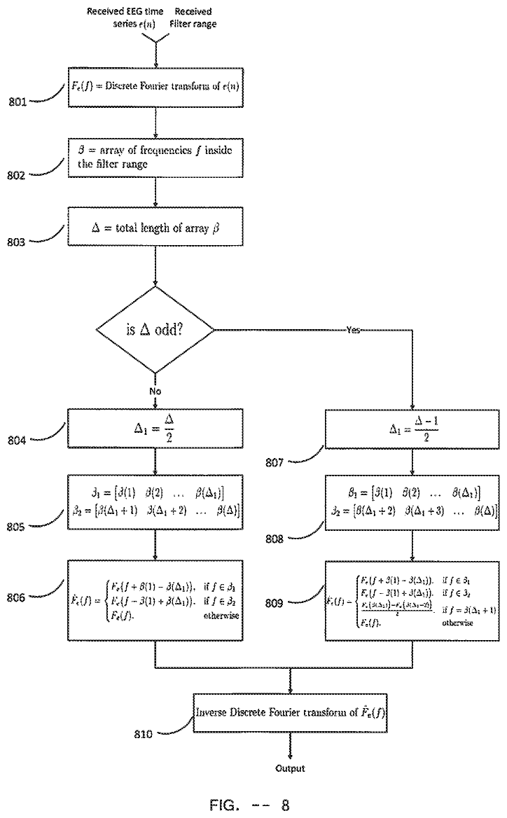

The Classification method 1010 takes the array of feature vectors from the Feature Extraction using LPC 1009 as input. It also takes PD Principal Components Array (PDPCA), PD Mean Array (PDMA), Healthy Principal Components Array (HPCA) and Healthy Mean Array (HMA) as inputs from the Classifier Data received by the Classifier Module. The Classification method 1010 implements the Classification Method Algorithm of FIG. 11 using (21-25) and generates a LEAPD index for each feature vector of the array of feature vectors from the Feature Extraction using LPC 1009 where each feature vector is obtained from EEG data of one subject. These LEAPD indices are delivered as an array of LEAPD indices in the output.

FIG. 11 shows a flow diagram of the Classification Method Algorithm which is used in the Classification method 1010 of FIG. 10. It generates a LEAPD index for a feature vector. The inputs to this algorithm are: a feature vector (denoted as a), PD Principal Components Array (PDPCA), PD Mean Array (PDMA), Healthy Principal Components Array (HPCA) and Healthy Mean Array (HMA). The steps of the Classification Method algorithm for generating LEAPD index from the given inputs are the following:
1. Calculate a_{PD} from (21)
2. Calculate a_{H} from (22)
3. Take each column of PDPCA and create the principal components of the PD subjects pₖ with that column
4. Calculate the distance vector D_{PD} using (23)
5. Take each column of HPCA and create the principal components of the healthy subjects hₖ with that column
6. Calculate the distance vector D_{H} using (24)
7. Calculate LEAPD index using (25)

After finding a LEAPD index using these steps, the calculated LEAPD index is delivered as output.

FIG. 12 shows a block diagram of a Cross Validation Fold Generator Module. It takes two inputs: Dataset and a Fold value. This Module randomly splits the received Dataset into multiple subsets and generates a collection of data-folds which is delivered as output. Given that the received Fold value is denoted as k and the received Dataset is denoted as S₀, it randomly splits S₀ into k number of subsets. Then it creates the data-folds by taking each subset Sᵢ as Test set and the rest of the remaining subsets as Training set. It executes the following steps to achieve this:
1. Randomly split S₀ into k disjoint subsets Sᵢ; i∈{1, 2, . . . , k} such that the ratio of PD Data and control Data in each subset Sᵢ is the same as in S₀, all Sᵢ have approximately the same size and the followings are true:

$$\begin{matrix}{S}_{i} ∈ {S}_{0} ; i ∈ \{ 1 , 2 , … , k \} & ( 46 ) \\ {S}_{i} ⋂ {S}_{j} = φ ; i \neq j and i , j \neq 0 & ( 47 ) \\ {⋃}_{i ∈ \{ 1 , 2 , … , k \} } {S}_{i} = {S}_{0} & ( 48 ) \\ \vert {S}_{j} \vert - 1 \leq \vert {S}_{i} \vert \leq \vert {S}_{j} \vert + 1 ; i , j ∈ \{ 1 , 2 , … , k \} and i \neq j & ( 49 )\end{matrix}$$

2. Create k number of data-folds where i-th data-fold is created by:

$${i}^{th} datafold = \{ \begin{matrix}Test set \text{:} & {S}_{i} \\ Training set \text{:} & {⋃}_{j ∈ \{ 1 , 2 , … , k \}}^{j \neq i} {S}_{j}\end{matrix} \qquad ( 50 )$$

3. Create a collection of the generated k number of data-folds and deliver as output.

FIG. 13 shows a block diagram of a Final Classification Data Generator Module. It takes two inputs: Dataset and Parameter Combination. It generates a Classifier Dataset as output.

The First Channel and its parameter 1306 selects the first element from the Parameter Combination received by the Final Classification Data Generator Module. It takes the Channel from the selected element of the Parameter Combination received by the Final Classification Data Generator Module and sets it as current Channel. It also takes the Parameter from the selected element of the Parameter Combination received by the Final Classification Data Generator Module and sets it as current Parameter. Then it delivers the current Parameter and the current Channel to the Classifier Data Generator 1311.

The Classifier Data Generator 1311 is a Classifier Data Generator Module of FIG. 6. It takes three inputs: Dataset, Parameters and Channel. The Dataset received by the Final Classification Data Generator Module is delivered to the Classifier Data Generator 1311 as Dataset. Current Parameter is delivered as Parameters and current Channel is delivered as Channel. It creates a Classifier Data for the current Channel and the current Parameter and delivers it as output for the Storage 1314.

The Storage 1314 takes the Classifier Data as input from the Classifier Data Generator 1311 and adds it in an array where each element of the array is a Classifier Data. The data structure of the Storage 1314 is shown in Table 1.

The decision block 1315 checks whether the current Channel and the current Parameter are from the last element of the Parameter Combination received by the Final Classification Data Generator Module. It the condition is false, Next parameter set 1316 selects the next element from the Parameter Combination received by the Final Classification Data Generator Module, takes the Channel from the selected element of the Parameter Combination and sets it as current Channel. Next parameter set 1316 also takes the Parameter from the selected element of the Parameter Combination received by the Final Classification Data Generator Module and sets it as the current Parameter. Then the current Parameter and the current Channel are delivered to the Classifier Data Generator 1311. Otherwise, Result 1317 outputs the Classifier Dataset.

The Result 1317 takes all Classifier data stored in the Storage 1314 and combines these Classifier Data into a Classifier Dataset which is delivered as Classifier Dataset in the output.

FIG. 14 shows a block diagram of a Final Classification Method Module. It takes two inputs: Classifier Dataset and an EEG Data. It calculates LEAPD index for the received EEG Data and delivers a LEAPD index as output.

The First Classifier Data 1404 takes the first Classifier Data in the Classifier Dataset received by the Final Classification Method Module, sets it as the current Classifier Data and passes it to the Classifier 1408.

The Classifier 1408 is a Classifier Module of FIG. 10. It takes two inputs: Dataset and Classifier Data. The EEG Data received by the Final Classification Method Module is delivered to the Classifier 1408 as Dataset and the current Classifier Data is delivered as Classifier Data. As the input Dataset of the Classifier 506 only has one EEG Data, it generates a single LEAPD index for the received EEG Data and delivers the LEAPD as output to the Storage 1412.

The Storage 1412 takes the LEAPD index as input from the Classifier 1408 and stores the LEAPD index in an array. When a new LEAPD index is delivered to the Storage 1412, it stores the new LEAPD index along with the previous LEAPD indices in the array.

The decision block 1413 checks whether the current Classifier Data is the last Classifier Data in the Classifier Dataset received by the Final Classification Method Module. If the condition is false, Next Classifier Data 1414 loads the next Classifier Data from the Classifier Dataset received by the Final Classification Method Module, sets it as the current Classifier Data and passes it to the Classifier 1408. Otherwise, the Result 1415 outputs a LEAPD index.

The Result 1415 takes all LEAPD indices stored in the array of the Storage 1412. It calculates the geometric mean of all of these LEAPD indices using (29) and delivers the geometric mean as a LEAPD index in the output.

To illustrate the advantages of the new method and system for diagnosis of Parkinson's disease according to the various implementations herein, it is necessary to provide comparisons with prior art methods. For this purpose, a dataset from New Mexico with 54 EEG data is used where 27 EEG data come from PD subjects and 27 EEG data come from healthy subjects. Also, for a separate out-of-sample test, a dataset from Iowa is used where 14 EEG data come from PD subjects and 14 EEG data come from healthy subjects. All healthy subjects are demographically matched for age and sex with PD subjects.

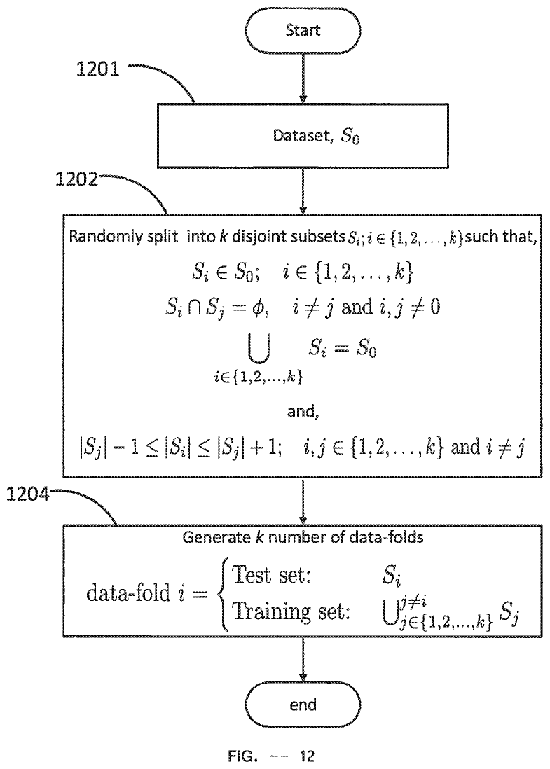

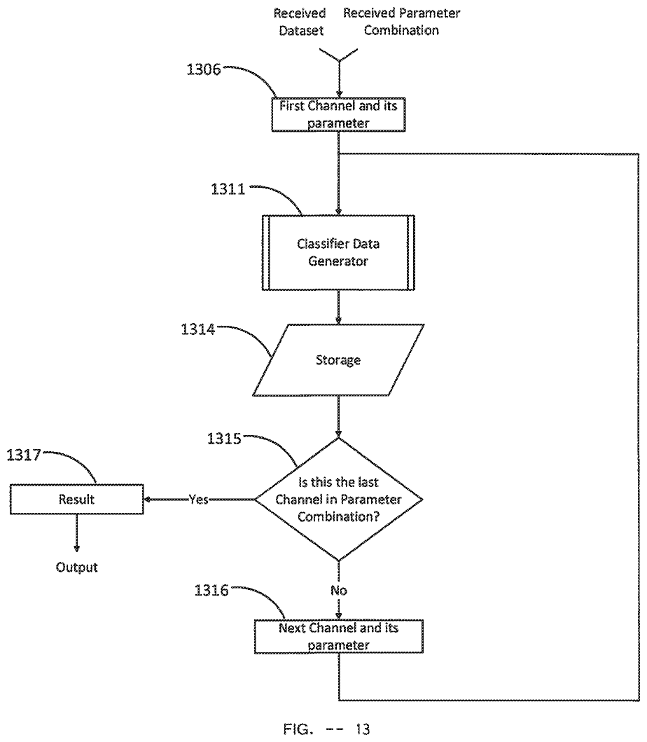

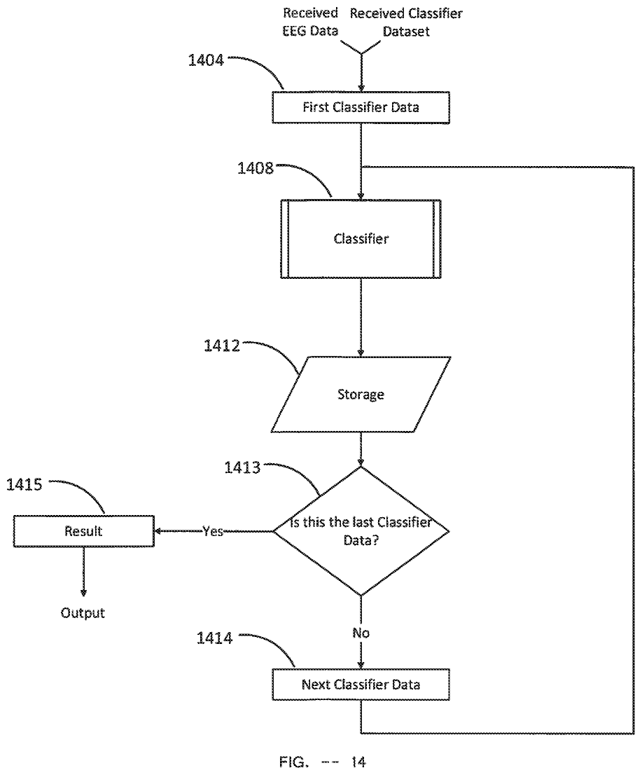

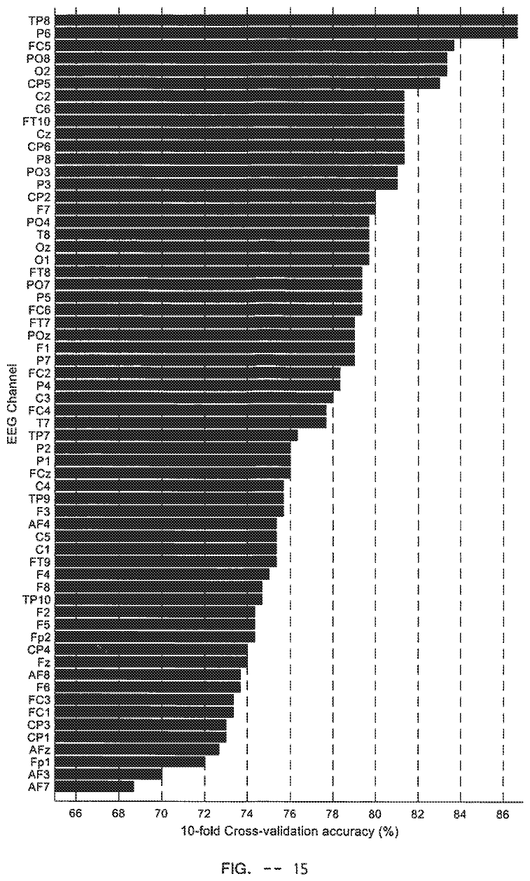

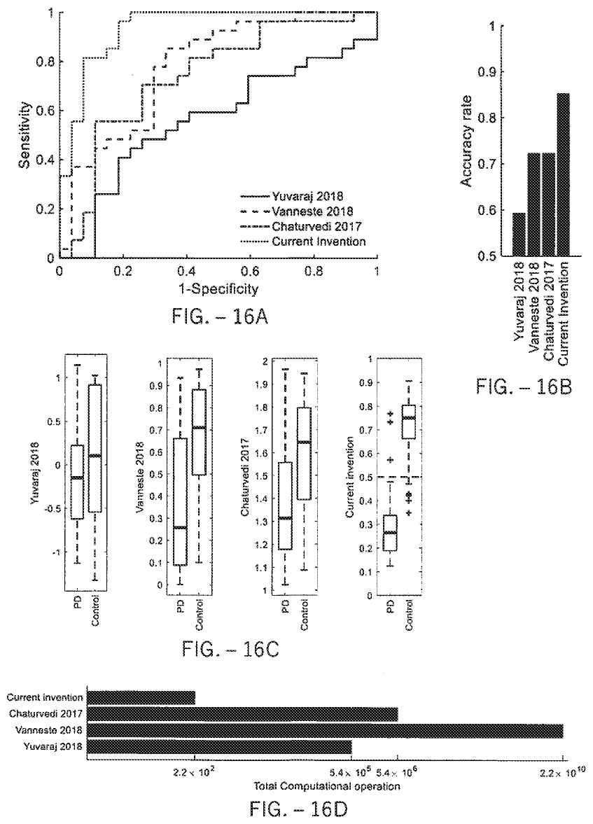

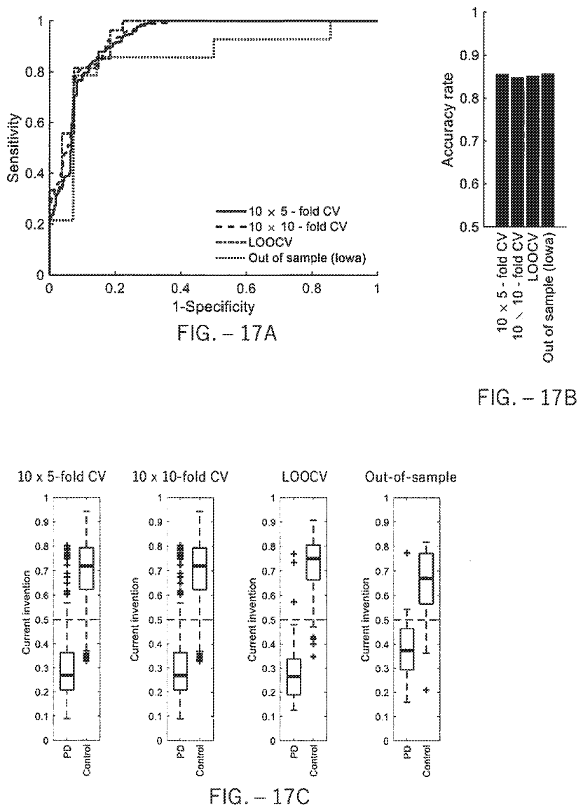

Prior art approaches can be compared with the disclosed system by using leave one out cross validation with the New Mexico dataset. FIG. 16A shows the receiver operator characteristics (ROC) curves comparison of the performance of various embodiments herein with the aforementioned methods which shows that the current system has significantly better ROC behavior. FIG. 16B shows the accuracy rate comparison for leave one out cross validation with New Mexico dataset and FIG. 16C shows the box plots of the outcomes from the aforementioned methods which illustrates that the LEAPD indices from the present implementation create clear separation between PD and healthy subjects and have better accuracy compared to the outcomes of the mentioned prior art. The techniques of the present system are computationally efficient and FIG. 16D shows a comparison of total computational operation for one classification where the various embodiments of the current system are clearly superior to the prior art. The performance of the current system is further tested with 10 rounds of 10-fold, 5-fold cross validations (CV) with New Mexico dataset and a separate out-of-sample test with Iowa dataset. FIG. 17A shows ROC curve comparison of these validation schemes and FIG. 17B shows the respective accuracy rates which illustrates that the performance of the various implementations is stable with multiple cross-validation schemes and even on an out-of-sample test. FIG. 17C box plots of the LEAPD indices on these validation schemes which shows a clear separation of LEAPD indices from PD and healthy subjects. The following table compares the methods and various validation schemes in terms of sensitivity (SEN), specificity (SPC), accuracy rate (ACC), positive predictive value (PPV), negative predictive value (NPV), odds ratio (OR), positive likelihood ratio (LR+) and area under the ROC curve (AUC). For each measurement, 95% Confidence Interval (95% CI) are also shown.

TABLE 7

| | Current system | | | | | | |
|---|---|---|---|---|---|---|---|
| | | | | Iowa | Prior art | | |
| | New Mexico | | | Out-of- | New Mexico (LOOCV) | | |
| | 10 × 5- | 10 × 10- | | sample | Chaturvedi | Vanneste | Yuvaraj et |
| | fold CV | fold CV | LOOCV | test | et al. [7] | et al. [10] | al. [8] |
| AUC % | 92.7 | 93.3 | 93.8 | 85.2 | 74.3 | 78.3 | 56.4 |
| | (90.5- | (91.2- | (87.4-100) | (69.5-100) | (60.8- | (66-90.7) | (40.6- |
| | 94.9) | 95.4) | | | 87.9) | | 72.1) |
| ACC % | 85.4 | 85.4 | 85.2 | 85.7 | 72.2 | 72.2 | 59.3 |
| | (82.1- | (82.1- | (72.9- | (67.3-96) | (58.4- | (58.4- | (45-72.4) |
| | 88.2) | 88.2) | 93.4) | | 83.5) | 83.5) | |
| SEN % | 87 | 87.8 | 88.9 | 85.7 | 74.1 | 70.4 | 63 |
| | (82.4- | (83.3- | (70.8- | (57.2-98.2) | (53.7- | (49.8- | (42.4- |
| | 90.8) | 91.4) | 97.6) | | 88.9) | 86.3) | 80.6) |
| SPC % | 83.7 | 83 | 81.5 | 85.7 | 70.4 | 74.1 | 55.7 |
| | (78.7- | (77.9- | (61.9- | (57.2-98.2) | (49.8- | (53.7- | (35.3- |
| | 87.9) | 87.2) | 93.7) | | 86.3) | 88.9) | 74.5) |
| PPV % | 84.2 | 83.7 | 82.8 | 85.7 | 71.4 | 73.1 | 58.6 |
| | (80.2- | (79.8- | (68.3- | (62-95.7) | (57.3- | (57.8- | (45.9- |
| | 87.5) | 87.1) | 91.5) | | 82.3) | 84.3) | 70.3) |
| NPV % | 86.6 | 87.2 | 88 | 85.7 | 73.1 | 71.4 | 60 |
| | (82.5- | (83.1- | (71.3- | (62-95.7) | (57.8- | (57.3- | (45.2- |
| | 89.8) | 90.4) | 95.6) | | 84.3) | 82.3) | 73.1) |
| OR | 34.5 | 35 | 35.2 | 36 | 6.8 | 6.8 | 2.1 |
| | (21.3- | (21.6- | (7.5- | (4.3-299) | (2.1-22.4) | (2.1-22.4) | (0.7-6.3) |
| | 55.7) | 56.7) | 164.8) | | | | |
| LR+ | 5.3 | 5.1 | 4.8 | 6 | 2.5 | 2.7 | 1.42 |
| | (4.1-7) | (3.9-6.7) | (2.1-10.7) | (1.6-22.3) | (1.3-4.7) | (1.4-5.4) | (0.8-2.4) |

Additionally, many of the prior art are highly sensitive to PD medications like Levodopa medication. FIG. 18A shows the boxplots of LEAPD indices of the same PD subjects with medication (Medication ON) and without medications (Medication OFF) along with the LEAPD indices of healthy controls.

FIG. 18B shows the accuracy performances of the current system on detecting PD subjects with (88.9%) and without medication (81.5%) and lastly, FIG. 18C shows a scatter plot of LEAPD indices with and without medication which shows a good correlation between the LEAPD indices of these two medication states. Finally, an insignificant p-value of 0.15 is found with the Univariate ANOVA test. These results illustrate that LEAPD index of the current system and method is not significantly sensitive to medication state which is not the case for the prior art. The exemplary embodiment herein is an advance for several reasons: (1) it shows a 13% increase in accuracy compared to other methods in a head-to-head test. While some of the studies reported moderate accuracy performance, there are high accuracies in past works but these prior methods included longer datasets, unbalanced classes (31 PD subjects and 264 controls for Vanneste et. al. [8]) across a wide age range (20-75 years) without age/sex-matched controls [7-9]. (2) The performances of the various system and method implementations herein are reliable among various schemes of cross validations with multiple rounds while prior works only performed one round of cross-validation. (3) The embodiments herein provide reliable performance in out-of-sample testing, which is missing in prior art. (5) LEAPD index of the current system and method is consistent despite the medication state, unlike prior art. (6) Finally, the current system and method is highly efficient and is amenable to real time applications. The aforementioned description of the various implementations herein has been presented for purposes of illustration and description which is not intended to be exhaustive or to limit those various implementations to the precise form disclosed. Many possible modifications and variations are possible in light of the above description. The non-limiting, exemplary embodiments herein are chosen for the best explanation of the principles of the various implementations herein and implementation thereof so that the other skilled in the art can utilize the various embodiments thereof.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “subject” refers to the target of administration, e.g., an animal. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed systems and methods, the subject has been diagnosed with a need for treatment of Parkinson's Disease prior to the treatment step.

Although the disclosure has been described with reference to preferred embodiments, persons skilled in the art will recognize that changes may be made in form and detail without departing from the spirit and scope of the disclosed apparatus, systems and methods.

What is claimed is:

1. A method for diagnosing Parkinson's Disease (PD) from electroencephalography (EEG) data comprising:
utilizing a system comprising:
(a) a computer processor for processing data; and
(b) a storage system for storing data on a storage medium;
receiving an electroencephalography (“EEG”) time series data for diagnosis;
calculating a Linear-predictive-coding Electroencephalogy Algorithm in PD (“LEAPD”) index for the EEG time series data; and
diagnosing a patient from the EEG time series data using the LEAPD index.

2. The method of claim 1, wherein the calculating the LEAPD index for the EEG time series data comprises:
filtering said EEG time series data with predetermined filter range;
determining Linear Predictive Coding (LPC) coefficients from the EEG time series data with predetermined order and creating feature vector a;
calculating a_PD and a_H from equations (21) and (22);
calculating the distance vector D_PD from PD Principal Components Array (“PDPCA”) using equation (23);
calculating the distance vector D_H from Healthy Principal Components Array (“HPCA”) using equation (24); and
calculating LEAPD index p using equation (25).

3. The method of claim 1, wherein the diagnosing the patient from the EEG time series data using the LEAPD index comprises:
generating Linear Predictive Coding (“LPC”) coefficients from the EEG time series data;
recognizing that vector of LPC coefficients for PD patients and healthy controls lie in separate hyperplanes;
finding the hyperplane for the PD patients and the hyperplane for the healthy controls;
calculating the distances between the vector created by the LPC coefficients and the PD patients hyperplane and the healthy controls hyperplane; and
determining whether the distance between the vector created by the LPC coefficients and the hyperplane for the PD patients is smaller than the distance between the vector created by the LPC coefficients and the hyperplane for the healthy controls, and if so, then diagnosing the patient as having PD.

4. The method of claim 1, wherein the diagnosing the patient from the EEG time series data using said LEAPD index comprises:
diagnosing the patient as healthy if the LEAPD index value is less than 0.5; and
diagnosing the patient as having PD if the LEAPD index value is greater than 0.5.

5. The method of claim 1, further comprising:
utilizing a training dataset of multiple pre-diagnosed EEG time series data and a predetermined value of filter range, Linear Predictive Coding (“LPC”) order and number of components;
filtering all EEG time series data of said training dataset with the predetermined filter range;
calculating feature vector by determining LPC coefficients for each EEG time series data of said training dataset using Burg's method with the predetermined order;
creating X_PD by combining the feature vectors of all EEG time series data from said training set pre-diagnosed as PD by using equation (9);
determining PD Mean Array (“PDMA”) by using equation (11);
determining a set of principal components from X_PD for PD;
finding PD Principal Components Array (“PDPCA”) by taking the predetermined number of components from the set of principal components;
creating X_H by combining the LPC coefficients of all EEG time series data from said training set pre-diagnosed as healthy by using equation (10);
determining Healthy Mean Array (“HMA”) by using equation (12);
determining a set of principal components from X_H for healthy; and
finding Healthy Principal Components Array (“HPCA”) by taking the predetermined number of components from the set of principal components from X_H for healthy.

6. A system for diagnosing Parkinson's Disease (“PD”) from electroencephalography (EEG) data comprising:
(a) a computer processing system comprising:
(i) a processor;
(ii) a storage medium associated with the processor;
(iii) hardware associated with the processor, the hardware configured to receive EEG time series data for diagnosis;
(b) a software module configured to calculate a Linear-predictive-coding Electroencephalogy Algorithm in PD (“LEAPD”) index for the EEG time series data; and
(c) a software module configured to diagnose a patient from the EEG time series data using said LEAPD index.

7. The system of claim 6, wherein the software module configured to calculate the LEAPD index comprises:
(a) a filtering step of the EEG time series data with predetermined filter range;
(b) a Burg's method step for determining LPC coefficients from said EEG time series data with predetermined order and creating feature vector a;
(c) a step for calculating a_PD and a_H from equation (21) and equation (22);
(d) a step for calculating the distance vector D_PD from PD Principal Components Array (“PDPCA”) using equation (23);
(e) a step for calculating the distance vector D_H from Healthy Principal Components Array (“HPCA”) using equation (24); and
(f) a step for calculating LEAPD index p using equation (25).

8. The system of claim 6, wherein the software module configured to diagnose the patient from the EEG time series data using said LEAPD index comprises:
(a) a Linear Predictive Coding (“LPC”) coefficients generating step from the EEG time series data;
(b) a step of recognizing that vector of LPC coefficients for PD patients and healthy controls lie in separate hyperplanes;
(c) a step of finding the hyperplane for the PD patients and the hyperplane for the healthy controls;
(d) a step of calculating the distances between the vector created by the LPC coefficients and the hyperplane for the PD patients and the hyperplane for the healthy controls; and
(e) a step of determining whether the distance between the vector created by the LPC coefficients and the hyperplane for the PD patients is smaller than the distance between the vector created by the LPC coefficients and the hyperplane for the healthy controls, and if so, then a step of diagnosing the patient as having PD.

9. The system of claim 6, wherein the software module configured to diagnose the patient from the EEG time series data using said LEAPD index comprises:
(a) a step of diagnosing the patient as healthy if the LEAPD index value is less than 0.5; and
(b) a step of diagnosing the patient as having PD if the LEAPD index value is greater than 0.5.

10. The system of claim 6, further comprising:
(a) a training dataset of multiple pre-diagnosed EEG time series data;
(b) a predetermined value of filter range, Linear Predictive Coding (LPC) order and number of components;
(c) a step of filtering all EEG time series data of said training dataset with the predetermined filter range;
(d) a step of calculating feature vector by determining LPC coefficients for each EEG time series data of said training dataset using Burg's method with the predetermined order;
(e) a step of creating X_PD by combining the feature vectors of all EEG time series data from said training set pre-diagnosed as PD by using equation (9);
(f) a step of determining PD Mean Array (“PDMA”) by using equation (11)
(g) a step of determining a set of principal components from X_PD for PD;
(h) a step for finding PD Principal Components Array (“PDPCA”) by taking the predetermined number of components from the set of principal components;
(i) a step of creating X_H by combining the LPC coefficients of all EEG time series data from said training set pre-diagnosed as healthy by using equation (10);
(j) a step of determining Healthy Mean Array (“HMA”) by using equation (12);
(k) a step of determining a set of principal components from X_H for healthy; and
(l) a step of finding Healthy Principal Components Array (“HPCA”) by taking the predetermined number of components from the set of principal components from X_H for healthy.

11. A PD EEG system comprising:
(a) a computer processor for processing data; and
(b) a storage system for storing data on a storage medium,
wherein the processor and storage system are configured for:
(i) receiving an EEG time series data;
(ii) calculating a LEAPD index for the EEG time series data.

12. The method of claim 11, wherein the calculating the LEAPD index for the EEG time series data comprises filtering said EEG time series data with predetermined filter range.

13. The method of claim 11, wherein the calculating the LEAPD index for the EEG time series data comprises determining LPC coefficients from the EEG time series data with predetermined order and creating feature vector a.

14. The method of claim 11, wherein the calculating the LEAPD index for the EEG time series data comprises calculating a_PD and a_H from equations (21) and (22).

15. The method of claim 14, wherein the calculating the LEAPD index for the EEG time series data comprises calculating the distance vector D_PD from PDPCA using equation (23).

16. The method of claim 15, wherein the calculating the LEAPD index for the EEG time series data comprises calculating the distance vector D_H from HPCA using equation (24).

17. The method of claim 11, wherein the calculating the LEAPD index for the EEG time series data comprises calculating LEAPD index p using equation (25).
